SEQUENCE LISTING (57 sequences)

SEQ ID NO: 1 - DNA, 4173 bp, Homo sapiens
  CDS (1)...(4170)
  sig_peptide (1)...(72)
  mat_peptide (73)...(4170)
atgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtcgtcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatag

SEQ ID NO: 2 - protein, 1390 aa, Homo sapiens [sequence omitted]

SEQ ID NO: 3 - DNA, 3252 bp, Homo sapiens
  CDS (1)...(3249)
tccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtcgtcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatag

SEQ ID NO: 4 - protein, 1083 aa, Homo sapiens
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVVLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 5 - protein, 40 aa, Homo sapiens
WHPKAEMRPSFSELVSRISAIFSTFIGEHYVHVNATYVNV

SEQ ID NO: 6 - DNA, 2187 bp, Homo sapiens
  CDS (1)...(2184)
atgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatag

SEQ ID NO: 7 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 8 - protein, 19 aa, Homo sapiens
  VARIANT 1: Xaa = Ser, Ala or other amino acid
XSESSASSDXXHPVITPSX

SEQ ID NO: 9 - DNA, 1512 bp, Homo sapiens
  CDS (1)...(1509)
atgacaacggacgacacagaagtgcccgctatgactctagcaccgggccacgccgctctggaaactcaaacgctgagcgctgagacctcttctagggcctcaaccccagccggccccattccagaagcagagaccaggggagccaagagaatttcccctgcaagagagaccaggagtttcacaaaaacatctcccaacttcatggtgctgatcgccacctccgtggagacatcagccgccagtggcagccccgagggagctggaatgaccacagttcagaccatcacaggcagtgatcccgaggaagccatctttgacaccctttgcaccgatgacagctctgaagaggcaaagacactcacaatggacatattgacattggctcacacctccacagaagctaagggcctgtcctcagagagcagtgcctcttccgacggcccccatccagtcatcaccccgtcacgggcctcagagagcagcgcctcttccgacggcccccatccagtcatcaccccgtcacgggcctcagagagcagcgcctcttccgacggcccccatccagtcatcaccccctcatggtccccgggatctgatgtcactctcctcgctgaagccctggtgactgtcacaaacatcgaggttattaattgcagcatcacagaaatagaaacaacaacttccagcatccctggggcctcagacatagatctcatccccacggaaggggtgaaggcctcgtccacctccgatccaccagctctgcctgactccactgaagcaaaaccacacatcactgaggtcacagcctctgccgagaccctgtccacagccggcaccacagagtcagctgcacctcatgccacggttgggaccccactccccactaacagcgccacagaaagagaagtgacagcacccggggccacgaccctcagtggagctctggtcacagttagcaggaatcccctggaagaaacctcagccctctctgttgagacaccaagttacgtcaaagtctcaggagcagctccggtctccatagaggctgggtcagcagtgggcaaaacaacttcctttgctgggagctctgcttcctcctacagcccctcggaagccgccctcaagaacttcaccccttcagagacaccgaccatggacatcgcaaccaaggggcccttccccaccagcagggaccctcttccttctgtccctccgactacaaccaacagcagccgagggacgaacagcaccttagccaagatcacaacctcagcgaagaccacgatgaagcccccaacagccacgcccacgactgcccggacgaggccgaccacagacgtgagtgcaggtgaaaatggaggtttcctcctcctgcggctgagtgtggcttccccggaagacctcactgaccccagagtggcagaaaggctgatgcagcagctccaccgggaactccacgcccacgcgcctcacttccaggtctccttactgcgtgtcaggagaggctaa

SEQ ID NO: 10 - protein, 503 aa, Homo sapiens
MTTDDTEVPAMTLAPGHAALETQTLSAETSSRASTPAGPIPEAETRGAKRISPARETRSFTKTSPNFMVLIATSVETSAASGSPEGAGMTTVQTITGSDPEEAIFDTLCTDDSSEEAKTLTMDILTLAHTSTEAKGLSSESSASSDGPHPVITPSRASESSASSDGPHPVITPSRASESSASSDGPHPVITPSWSPGSDVTLLAEALVTVTNIEVINCSITEIETTTSSIPGASDIDLIPTEGVKASSTSDPPALPDSTEAKPHITEVTASAETLSTAGTTESAAPHATVGTPLPTNSATEREVTAPGATTLSGALVTVSRNPLEETSALSVETPSYVKVSGAAPVSIEAGSAVGKTTSFAGSSASSYSPSEAALKNFTPSETPTMDIATKGPFPTSRDPLPSVPPTTTNSSRGTNSTLAKITTSAKTTMKPPTATPTTARTRPTTDVSAGENGGFLLLRLSVASPEDLTDPRVAERLMQQLHRELHAHAPHFQVSLLRVRRG

SEQ ID NO: 11 - DNA, 1971 bp, Mus musculus
  CDS (1)...(1968)
atgggctctgtctggggcctggctgtgcccctccttgtcttctgctggaaggttggcgtcagtgtgagctctccaggtctcgacattagcagatcagttcctttggtgacaaccaacaacatggaagtgtccaccttcactcaaagggacaggcccagctcagagagagctttccagaccaccaacctgattcagtatgtacctctagacacacaaactctcagcactgaatctgcttctaatgctttaagtgcaactagtatcagttcagaggtcaattccagagacactcaggccacctcttttgtaacaaagaccaggaagacacatactaccactcctgcagcatcatccctagaggcccagaccacctctcctaatttgtcaaccctgaatattcagattacctccccaatagcatcgtccctggatgctcagaccatttttcctgtatcattatccctgagtacccagaccacttctcctgcaccatcattcctggatacccaaaccacttcccctgaaccatcatctctgaccacttcccctgcaccatcatctctgatcacttctcctactccatcatctctaactatatcccctgcaccgtcattcctggatacccagaccacttcccctgcactatcatctctggctacttcccctgcaccatcatccctagatacccagaccatctcgcctattgaattgaccctaaagacccaaaccatttccactgtaacggagaccagaacggtttcaataagaataccttctgacctcaaggttatgcacaccatccctacagagacattgacaccaagcaacagccccagaactgggatgagcactgtccaaactggcacagtgtgggaccccatagaagccatctttgacaccctttgtaccgatgacagctctgaagaggcaaggaagatcacagttgacctcctgacgttggcacacacctccacggaagtggagcacctgtcttcagagagcagctcttcctctgacagttcagctggggtcctcagcagctcacgagtcctgggacccgacagtgcaactccagccaaggggttggttgccttcaacatcactcatattaaactcagcacctgcatcacagaaatagaaacaaccatcaccatctctggggccccaggcgtgagcctcagccctacagaagcgacggcagctttgttcacttctgagatattaactttgcctccacccactgaggcaaaaccaattttccccgagaccacaagcttatctgggatcctgtcaactgccggcaccccggcccttgccaccacccttgaggggacagtctccactagtgcaatcacagaaagcgaaacagcagtggcccagactcttacctctgttggaacttcggtgacagttagaaggaaccccctggaaaacacttcaacactctctattgagacacaaagccacactgaggtcttgggcaccatcacagtccccatggttgctggatcaacaatgggagaaacagcctccttcgtgagtttcacggctctcgacagcagcagtctctcagtggtggtcaccaccgagagctctgctacctcagagactttaaccacaggcaatacaaccaacagctccttcctcacagaaagccaccctcctttttctatctattcaactacagctagcaccagcaaaaacccaaacatcaccttaaccaagaccacagcctcaccaaagcccccgacgcatcctacaacatctgcatccactgcttggataaggaaaaccacaaaacatgatccaggtgaagatggaggcttcctccttgtacggctgaccgtggcctcccctaaggacctcactgagcacaacgccagagagaagctgatgaaccagctccgccgtgaactgcacgcccgcatgccacttgtccacatgtccttcctgagcatcaggaggggttga

SEQ ID NO: 12 - protein, 656 aa, Mus musculus
MGSVWGLAVPLLVFCWKVGVSVSSPGLDISRSVPLVTTNNMEVSTFTQRDRPSSERAFQTTNLIQYVPLDTQTLSTESASNALSATSISSEVNSRDTQATSFVTKTRKTHTTTPAASSLEAQTTSPNLSTLNIQITSPIASSLDAQTIFPVSLSLSTQTTSPAPSFLDTQTTSPEPSSLTTSPAPSSLITSPTPSSLTISPAPSFLDTQTTSPALSSLATSPAPSSLDTQTISPIELTLKTQTISTVTETRTVSIRIPSDLKVMHTIPTETLTPSNSPRTGMSTVQTGTVWDPIEAIFDTLCTDDSSEEARKITVDLLTLAHTSTEVEHLSSESSSSSDSSAGVLSSSRVLGPDSATPAKGLVAFNITHIKLSTCITEIETTITISGAPGVSLSPTEATAALFTSEILTLPPPTEAKPIFPETTSLSGILSTAGTPALATTLEGTVSTSAITESETAVAQTLTSVGTSVTVRRNPLENTSTLSIETQSHTEVLGTITVPMVAGSTMGETASFVSFTALDSSSLSVVVTTESSATSETLTTGNTTNSSFLTESHPPFSIYSTTASTSKNPNITLTKTTASPKPPTHPTTSASTAWIRKTTKHDPGEDGGFLLVRLTVASPKDLTEHNAREKLMNQLRRELHARMPLVHMSFLSIRRG

SEQ ID NO: 13 - DNA, 491 bp, Rattus norvegicus
  CDS (3)...(488)
ctgaggtttttggtaccatcacagtccccacggttgctggatcaacagtaggagaagcaacctctcttgtgagttccacagctctagacagcagtctctcagcagtggtcaccaccaagggctctgcttcctcagagactttaaccacagacaatacaaccaacagctccttcctcacagggagtcgccctccttctttaatctattcgacaacagctagcaccagtgaaagaacaaacgtcactttaaccaagaccacagcctcaccgaagaccccgatgaatcccgcacccactgcttggacaaggaaaaccacagagcacgatccaggtataaatggaggcttcctccttgtacggctgaccgtggcctcccctaaggacctcactgagcacgtcaccagagagaagctgatgcaccagctccgccgtgaactgcacacccatatgccacttgtccaagtgtccttactgagcatcaggaggagctga

SEQ ID NO: 14 - protein, 162 aa, Rattus norvegicus
EVFGTITVPTVAGSTVGEATSLVSSTALDSSLSAVVTTKGSASSETLTTDNTTNSSFLTGSRPPSLIYSTTASTSERTNVTLTKTTASPKTPMNPAPTAWTRKTTEHDPGINGGFLLVRLTVASPKDLTEHVTREKLMHQLRRELHTHMPLVQVSLLSIRRS

SEQ ID NO: 15 - protein, 53 aa, Homo sapiens
GENGGFLLLRLSVASPEDLTDPRVAERLMQQLHRELHAHAPHFQVSLLRVRRG

SEQ ID NO: 16 - protein, 993 aa, Escherichia coli
MTMITDSLAVVARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 17 - protein, 789 aa, Escherichia coli
MTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAP

SEQ ID NO: 18 - DNA, 31 bp, Artificial Sequence
  PCR primer
ttcagaattctcagagacaccgaccatggac

SEQ ID NO: 19 - DNA, 28 bp, Artificial Sequence
  PCR primer
cacggaattcactgcccggacgaggccg

SEQ ID NO: 20 - DNA, 31 bp, Artificial Sequence
  PCR primer
gggggtcgacttagcctctcctgacacgcag

SEQ ID NO: 21 - protein, 17 aa, Homo sapiens
RGAKRISPARETRSFTK

SEQ ID NO: 22 - DNA, 32 bp, Artificial Sequence
  PCR primer
ggggaagcttatgacaacggacgacacagaag

SEQ ID NO: 23 - DNA, 60 bp, Artificial Sequence
  PCR primer
aaaactcgagtcacttgtcgtcatcgtctttgtagtcgcctctcctgacacgcagtaagg

SEQ ID NO: 24 - DNA, 54 bp, Artificial Sequence
  PCR primer
aaaactcgagtcaatggtgatggtgatgatggcctctcctgacacgcagtaagg

SEQ ID NO: 25 - DNA, 21 bp, Artificial Sequence
  PCR primer
cagagtggcagaaaggctgat

SEQ ID NO: 26 - DNA, 20 bp, Artificial Sequence
  PCR primer
ggaagcacacagatgggtga

SEQ ID NO: 27 - DNA, 30 bp, Artificial Sequence
  PCR primer
aaaagaattcgtgaaggcctcgtccacctc

SEQ ID NO: 28 - DNA, 31 bp, Artificial Sequence
  PCR primer
aaaagtcgacttagcctctcctgacacgcag

SEQ ID NO: 29 - DNA, 30 bp, Artificial Sequence; identical to SEQ ID NO: 27
  PCR primer

SEQ ID NO: 30 - DNA, 33 bp, Artificial Sequence
  PCR primer
ctccgtcgacttatgcactcacgtctgtggtcg

SEQ ID NO: 31 - DNA, 32 bp, Artificial Sequence
  PCR primer
aaaagaattcggtgaaaatggaggtttcctcc

SEQ ID NO: 32 - DNA, 31 bp, Artificial Sequence; identical to SEQ ID NO: 28
  PCR primer

SEQ ID NO: 33 - DNA, 37 bp, Artificial Sequence
  PCR primer
ggggctcgagatgaccatgattacggattcactggcc

SEQ ID NO: 34 - DNA, 35 bp, Artificial Sequence
  PCR primer
gggggtcgacttatttttgacaccagaccaactgg

SEQ ID NO: 35 - DNA, 37 bp, Artificial Sequence; identical to SEQ ID NO: 33
  PCR primer

SEQ ID NO: 36 - DNA, 35 bp, Artificial Sequence
  PCR primer
gggggtcgacttacggtgcacgggtgaactgatcg

SEQ ID NO: 37 - DNA, 32 bp, Artificial Sequence; identical to SEQ ID NO: 22
  PCR primer

SEQ ID NO: 38 - DNA, 33 bp, Artificial Sequence
  PCR primer
aaaactcgaggcctctcctgacacgcagtaagg

SEQ ID NO: 39 - DNA, 32 bp, Artificial Sequence; identical to SEQ ID NO: 22
  PCR primer

SEQ ID NO: 40 - DNA, 31 bp, Artificial Sequence
  PCR primer
cattctcgagtgcactcacgtctgtggtcgg

SEQ ID NO: 41 - DNA, 37 bp, Artificial Sequence
  PCR primer
gtttaagcttatgagaagactcctacaacccgaatac

SEQ ID NO: 42 - DNA, 31 bp, Artificial Sequence
  PCR primer
actactcgagtgatgtctcccagaaggaggc

SEQ ID NO: 43 - DNA, 25 bp, Artificial Sequence
  PCR primer
catccaagccatatatggacgttcc

SEQ ID NO: 44 - DNA, 25 bp, Artificial Sequence
  PCR primer
tctggagagtcaaaattctcttcgt

SEQ ID NO: 45 - DNA, 23 bp, Artificial Sequence
  PCR primer
ggcatccggcacctctatggtcc

SEQ ID NO: 46 - DNA, 24 bp, Artificial Sequence
  PCR primer
gccacttgtcggcgataaggaagg

SEQ ID NO: 47 - DNA, 26 bp, Artificial Sequence
  PCR primer
tgaaggtcggagtcaacggatttggt

SEQ ID NO: 48 - DNA, 24 bp, Artificial Sequence
  PCR primer
catgtgggccatgaggtccaccac

SEQ ID NO: 49 - DNA, 21 bp, Artificial Sequence
  PCR primer
atgctgtggatctgggctgtc

SEQ ID NO: 50 - DNA, 21 bp, Artificial Sequence
  PCR primer
ggttctagattcagggtcatc

SEQ ID NO: 51 - DNA, 29 bp, Artificial Sequence
  PCR primer
gcatctagaatcgaggttattaattgcag

SEQ ID NO: 52 - DNA, 21 bp, Artificial Sequence
  PCR primer
ttagcctctcctgacacgcag

SEQ ID NO: 53 - DNA, 39 bp, Artificial Sequence
  PCR primer
aaaaaagcttggcaccatgctgaaggcctcgtccacctc

SEQ ID NO: 54 - DNA, 31 bp, Artificial Sequence
  PCR primer
aaaactcgagttacggagctgctcctgagac

SEQ ID NO: 55 - DNA, 33 bp, Artificial Sequence
  PCR primer
aaaactcgagttaaggggtgaagttcttgaggg

SEQ ID NO: 56 - DNA, 32 bp, Artificial Sequence
  PCR primer
aaaactcgagttacgtccctcggctgctgttg

SEQ ID NO: 57 - DNA, 32 bp, Artificial Sequence
  PCR primer
aaaactcgagttatgcactcacgtctgtggtc

(19) United States
(12) Patent Application Publication
(10) Pub. No.: US 2005/0113284 A1
(43) Pub. Date: May 26, 2005

(54) Drug for regenerating tissue and vessel and method therefor

(76) Inventors: Motonao Nakamura, Kanagawa (JP); Toshio Higuchi, Kanagawa (JP); Yoshiki Yamasaki, Kanagawa (JP); Takuya Orita, Kanagawa (JP)

Correspondence Address: FISH & RICHARDSON PC, 225 FRANKLIN ST, BOSTON, MA, 02110, US

(21) Appl. No.: 10/498,332

(22) PCT Filed: Dec. 12, 2002
(86) PCT No.: PCT/JP02/13014

(30) Foreign Application Priority Data
Dec. 13, 2001 (JP) 2001-380158
Dec. 4, 2002 (JP) 2002-352924

(51) Int. Cl. A61K 38/17; A61K 48/00

(52) U.S. Cl. 514/2; 514/44

(57) ABSTRACT

The present invention revealed that the functional expression of hepatocyte growth factor (HGF)/HGF receptor is suppressed through the binding of the HGF receptor to a novel mucin-like protein, leading to the inhibition of HGF dependent cell proliferation and regeneration/neogenesis of tissues and blood vessels. Furthermore, inhibition of the binding between the HGF receptor and the mucin-like protein was found to enable treatment of various diseases including liver diseases.

### TECHNICAL FIELD

The present invention relates to pharmaceutical compositions comprising a substance having activity to inhibit the biological activity of c-Met Regulatory Mucin (MERMUC). Specifically, the present invention relates to a substance having activity to inhibit the binding between MERMUC and c-Met or the expression of MERMUC.

More specifically, the invention relates to pharmaceutical compositions comprising a substance having activity to inhibit the binding between MERMUC and c-Met or the expression of MERMUC yet used for maintaining or enhancing cell proliferation or formation, repair, regeneration or neogenesis of tissues, organs, blood vessels, mucosa, skeleton or nerves due to hepatocyte growth factor (HGF).

### BACKGROUND ART

The living body of mammals and other organisms has regenerative ability to quickly repair or newly generate damaged or defective tissues and organs in vivo so that they function normally. In other words, this regenerative ability is indispensable for treating and recovering a living body from a diseased state.

Hepatocyte growth factor (HGF) has been extensively studied as one of the factors greatly contributing to this regenerative ability of tissues and organs. Furthermore, in the field of regenerative medicine therapy, HGF is greatly expected to enable treatment of various intractable diseases, and research and development for clinical applications is now being extensively pursued.

In 1984, HGF was identified as a potent growth factor of primary cultured hepatocytes partially purified from rat blood by Nakamura et al. (Biochem. Biophys. Res. Commun., 1984, Vol.122, p.1450-1459). Five years later in 1989, cDNAs encoding human and rat HGF were cloned and the primary structure of HGF was elucidated (Nature, 1989, Vol.342, p.440-443).

Human HGF is synthesized as a single-stranded inactive prepro form comprising 728 amino acid residues. Then, it is processed by a serine protease called HGF converting enzyme or HGF activator into an active heterodimer consisting of an α chain (molecular weight 69 kDa) and a β chain (molecular weight 34 kDa). This active heterodimer binds to the HGF receptor to exhibit various biological activities of HGF.

On the other hand, HGF was found to be the ligand for receptor-type tyrosine kinase c-Met that is a product of the c-met gene originally identified as an oncogene from human osteosarcoma cells. Thus, c-Met was identified to be the receptor for HGF (Science, 1991, Vol.251, p.801-804; Oncogene, 1991, Vol.6, p.501-504).

c-Met, which is an HGF receptor, is a heterodimer of disulfide-bonded α chain (molecular weight 50 kDa) and β chain (molecular weight 145 kDa). The β chain of c-Met comprises an extracellular region, transmembrane region and cytoplasmic region, and a tyrosine kinase domain exists in the cytoplasmic region. When HGF binds to receptor c-Met, the receptor dimerizes, and then autophosphorylation of the tyrosine residues existing in the cytoplasmic region is induced.

Furthermore, a site consisting of 3 phosphorylated tyrosine residues called multifunctional docking site exists on the C-terminal side of the c-Met β chain. Through the binding of various signal transduction molecules comprising an SH2 domain, such as PI3 kinase, PLC-γ, SHC, Grb-1, Grb-2 and pp60^{c-SRC}, to this site, transmission of signals involved in various biological activities of HGF is induced (for example, Cell, 1994, Vol.77, p.261-271, 1994; Jikken Igaku (Experimental Medicine), 1997, Vol.15, p.1033-1039).

Previous studies have revealed that HGF exhibits various kinds of biological activities, and therapeutic and ameliorative effects against disorders, as described below, through signal transduction into cells via the binding to its receptor, c-Met:
(1) HGF functions as a growth-promoting factor of not only hepatocytes but also most epithelial cells (renal tubule epithelial cells, skin keratinocytes, melanocytes, alveolar type II epithelial cells, gastric mucosal epithelial cells, etc.) and some mesenchymal cells (vascular endothelial cells, chondrocytes, osteoclasts, muscular satellite cells, etc.) (for example, J. Biochem., 1996, Vol.119, p.591-600; Biochem. Biophys. Res. Commun., 1997, Vol.239, p.639-644);
(2) HGF has the function as a mediator molecule for the interaction between epithelial cells and mesenchymal tissues in the developmental process of mammalian and amphibian organisms, and induces the formation of internal organs, such as liver, kidney and lung, and various tissue organs, such as limb bud and somite (J. Biochem., 1996, Vol.119, p.591-600; Biochem. Biophys. Res. Commun., 1997, Vol.239, p.639-644; J. Cell Biol., 1993, Vol.123, p.223-236; Development, 1998, Vol.125, p.1315-1324; Nature, 1995, Vol.373, p.699-702; Nature, 1995, Vol.373, p.702-705; Nature, 1995, Vol.376, p.768-771; Biochem. Biophys. Res. Commun., 1997, Vol.234, p.8-14);
(3) On the other hand, in a mature organism, HGF functions as a factor that promotes repair, regeneration and neogenesis of organs, such as liver, kidney and lung, and tissues thereof by functioning paracrine and endocrine (for example, J. Biochem., 1996, Vol.119, p.591-600; Biochem. Biophys. Res. Commun., 1997, Vol.239, p.639-644; J. Cell Biol., 1993, Vol.123, p.223-236; Biochem. Biophys. Res. Commun., 1990, Vol.173, p.42-47; Biochem. Biophys. Res. Commun., 1991, Vol.177, p.330-335; Hepatology, 1992, Vol.16, p.1227-1235; J. Biol. Chem., 1991, Vol.266, p.22781-22784; Am. J. Physiol., 1993, Vol.265, p.61-69; Proc. Natl. Acad. Sci. USA., 1994, Vol.91, p.4357-4361; Am. J. Physiol., 1996, Vol.270, p.1031-1039; Protein Nucleic Acid and Enzyme, 2000, Vol.45, No.13, p.2100-2108);
(4) Apart from the role in the proliferation of the above-mentioned epithelial cells and some mesenchymal cells (vascular endothelial cells, muscular cells, etc.), and the repair and regeneration of damages in organs such as liver, kidneys and lungs, HGF strongly promotes proliferation and repair of other cells, such as gastric mucosal epithelial cells, myocardial cells, skeletal muscle cells and nerve cells, and takes part in the prevention of disruption as well as regeneration and repair of gastric mucosa, heart, skeletal tissue and nervous tissue (for example, Protein Nucleic Acid and Enzyme, 2000, Vol.45, No.13, p.2100-2108);
(5) HGF suppresses apoptosis of vascular endothelial cells due to hyperglycemia, ischemia, etc. (for example, Diabetologia, 1997, Vol.40, p.1053-1061; Diabetes, 1997, Vol.46, p.138-142);
(6) Since the expression of uPA, MMP-1, MMP-9 and such are induced by stimulating myocardial fibroblasts with HGF, HGF is suggested to have an anti-fibrosis effect on various tissues (for example, Nihon Rinsho (Japanese Journal of Clinical Medicine), 2000, Vol.58, Suppl. p.168-172);
(7) Vascular neogenesis due to HGF has been confirmed by administering HGF or HGF gene to rabbits and rats (for example, Nihon Rinsho (Japanese Journal of Clinical Medicine, 2000, Vol.58, Suppl. p.168-172; Hypertension, 1999, Vol.33, p.1379-1384); and
(8) Animal testing has shown that HGF exhibits dramatic therapeutic effects towards intractable organ failure for which ultimate therapy still do not exist, such as hepatic fibrosis, hepatic cirrhosis, fulminant hepatitis, viral hepatitis, acute hepatitis, chronic renal failure, renal fibrosis and pulmonary fibrosis, through the wide variety of physiological activities of HGF described above (for example, Experimental Medicine, 1997, Vol.15, p.90-97; J. Biochem., 1995, Vol.118, p.643-649; Nature Med., 1999, Vol.5, p.226-230; Hepatology, 1997, Vol.26, p.81-89; Hepatology, 1999, Vol.30, p.151-159; Biochem. Biophys. Res. Commun., 1998, Vol.244, p.683-690; J. Clin. Invest., 1999, Vol.103, p.313-320; Kidney Int., 2000, Vol.57, p.937-948; J. Clin. Invest., 1998, Vol.101, p.1827-1834; Am. J. Repair. Crit. Care Med., 1997, Vol.156, p.1937-1944).

Furthermore, recently, treatment of arteriosclerosis obliterans by gene therapy using the HGF gene has been attempted.

While various biological activities and therapeutic effects of HGF that are effective for the treatment of various kinds of intractable diseases as mentioned above are elucidated, the possibility of carcinogenesis attributable to excessive provision of HGF into the patient's body is a matter of concern in the treatment of diseases using HGF or the HGF gene. Therefore, there is a demand for the development of novel methods that enable treatment of such diseases without depending only on the administration of HGF or HGF gene to the living body.

As an approach for developing such novel therapeutic methods, a promising method is to identify a novel molecule that modulates the HGF/c-Met signal transduction by interacting with c-Met, the HGF receptor, and modulate the interaction of the novel molecule with c-Met or the function of the novel molecule.

### DISCLOSURE OF THE INVENTION

Specifically, the objectives of the present invention are: (1) identifying a novel molecule that modulates the HGF/c-Met signal transduction by interacting with the HGF receptor c-Met; (2) providing methods, substances and pharmaceutical compositions that modulate the HGF/c-Met signal transduction by regulating the interaction of the novel molecule with c-Met or the function of the novel molecule; and (3) using these methods, substances and pharmaceutical compositions, to treat various diseases to which HGF drugs (HGF protein drugs, HGF gene therapy, etc.) are applied for treatment; in particular, intractable organ diseases such as hepatic fibrosis, hepatic cirrhosis, fulminant hepatitis, viral hepatitis, acute hepatitis, chronic renal failure, renal fibrosis and pulmonary fibrosis.

The present inventors extensively studied a recently reported mucin-like protein (International Application No. WO 01/05803) cloned from the renal tissue of a hepatitis patient (further described below) Specifically, molecules that bind to the mucin-like protein were studied.

As a result, the present inventors discovered that: (1) the HGF receptor c-Met binds to the mucin-like protein; (2) the binding of the two molecules suppressively regulates (down-regulates) the biological activity of HGF exhibited via the signal transduction mediated by HGF/c-Met; and (3) the binding affinity of the mucin-like protein to c-Met is increased by multimerization (self-association) of the mucin-like protein. Finally, the present invention was accomplished and will be described in detail below.

The present inventors dubbed this molecule “c-Met Regulatory Mucin (MERMUC)” based on these characteristics of the mucin-like protein.

The finding that the expression of the biological activity of HGF is down-regulated by the binding of its receptor c-Met with MERMUC (including self-associated MERMUC molecules), is obviously an unprecedented and completely novel finding. Furthermore, most importantly, based on this completely novel finding, the present invention that is extremely useful in medicine and pharmaceutical industries as described below was accomplished.

Specifically, this invention enables repair, regeneration or neogenesis of tissues and organs required for the prevention and treatment of various diseases using the minimum required amount of HGF drugs (HGF protein, HGF gene, etc.) or without such drugs. More specifically, the present invention enables treatment of various intractable diseases that are still considered difficult to treat (for example, hepatic fibrosis, hepatic cirrhosis, fulminant hepatitis, viral hepatitis, acute hepatitis, chronic renal failure, renal fibrosis and pulmonary fibrosis).

This is because the potential biological activity possessed by endogenous HGF or HGF exogenously supplied (as HGF protein drug or by HGF gene therapy) to the patient can be maximally drawn out through the elimination of HGF activity down-regulation that occurs due to the binding between c-Met and MERMUC using a pharmaceutical composition of the present invention. Such pharmaceutical compositions, specifically include: (1) those comprising a substance that has activity to inhibit the binding between MERMUC and the HGF receptor c-Met; (2) those comprising a substance that has activity to inhibit multimerization (self-association) of MERMUC molecules; and (3) those comprising a substance that suppresses or inhibits the expression of MERMUC.

Therefore, in the treatment of diseases using HGF drugs, the dose of the HGF drug administered to a patient, frequency of the administration and duration of the administration can be drastically decreased by combining a pharmaceutical composition of the present invention such as those described above. Furthermore, thereby, mental and physical pain of the patient can be reduced, and simultaneously, various costs (costs borne by each of patients, medical institutes, drug makers and government agencies) will be saved.

In addition to the above-mentioned pharmaceutical compositions, the present invention provides methods for identifying the active ingredients of the pharmaceutical compositions: (1) substances having activity to inhibit the binding between c-Met and MERMUC; (2) substances having activity to inhibit multimerization (self-association) of MERMUC; and (3) substances that suppress or inhibit the expression of MERMUC. Furthermore, the present invention provides methods of testing a given substance for various activities, such as: (1) activity to inhibit the binding between c-Met and MERMUC; and (2) activity to inhibit multimerization (self-association) of MERMUC. Moreover, the present invention provides various tools (various polypeptides, fusion polypeptides, etc.) used for these methods. The above-described pharmaceutical compositions of the present invention can be provided through these inventions of methods and tools.

More specifically, the present invention relates to:
(1) a pharmaceutical composition used for maintaining or enhancing cell proliferation, or formation, repair, regeneration or neogenesis of tissues, organs, blood vessels, mucosa, skeleton or nerves, which comprises a substance having activity to inhibit the binding between c-Met and MERMUC or multimerization of MERMUC, and a pharmaceutically acceptable carrier;
(2) a pharmaceutical composition used for maintaining or enhancing cell proliferation, or formation, repair, regeneration or neogenesis of tissues, organs, blood vessels, mucosa, skeleton or nerves due to HGF, which comprises a substance having activity to inhibit the binding between c-Met and MERMUC or multimerization of MERMUC, and a pharmaceutically acceptable carrier;
(3) the pharmaceutical composition of (2), wherein said HGF is an endogenous HGF of an organism;
(4) the pharmaceutical composition of (2), wherein said HGF is an HGF drug that exogenously provides HGF to an organism;
(5) the pharmaceutical composition of (4), wherein the HGF drug is a protein drug comprising an HGF protein;
(6) the pharmaceutical composition of (4), wherein the HGF drug is a DNA drug comprising DNA encoding an HGF protein;
(7) the pharmaceutical composition of (1) or (2), which is used for preventing or treating fibrosis;
(8) the pharmaceutical composition of (7), wherein the fibrosis is hepatic, renal or pulmonary fibrosis;
(9) the pharmaceutical composition of (1) or (2), which is used for preventing or treating hepatic cirrhosis, hepatic fibrosis or hepatitis;
(10) the pharmaceutical composition of (1) or (2), wherein hepatitis is used for preventing or treating fulminant hepatitis, acute hepatitis or viral hepatitis;
(11) the pharmaceutical composition of (1) or (2), which is used for preventing or treating chronic renal failure;
(12) the pharmaceutical composition of (1) or (2), which is used for preventing or treating arteriosclerosis obliterans;
(13) the pharmaceutical composition of (1) or (2), wherein the substance has activity to inhibit the binding between c-Met and MERMUC;
(14) the pharmaceutical composition of (13), wherein the substance has activity to inhibit the binding between the C-terminal region of the c-Met β chain and the C-terminal region of MERMUC;
(15) the pharmaceutical composition of (1) or (2), wherein the substance has activity to inhibit multimerization of MERMUC;
(16) the pharmaceutical composition of (1) or (2), wherein the substance binds to MERMUC;
(17) a pharmaceutical composition used for maintaining or enhancing cell proliferation, or formation, repair, regeneration or neogenesis of tissues, organs, blood vessels, mucosa, skeleton or nerves, which comprises a substance that suppresses or inhibits the expression of MERUMUC and a pharmaceutically acceptable carrier;
(18) the pharmaceutical composition of (17), wherein the substance inhibits the transcription of a gene encoding MERMUC to mRNA;
(19) the pharmaceutical composition of (17), wherein the substance inhibits the translation of an mRNA encoding MERMUC to protein;
(20) a pharmaceutical composition used for maintaining or enhancing cell proliferation, or formation, repair, regeneration or neogenesis of tissues, organs, blood vessels, mucosa, skeleton or nerves due to HGF, which comprises a substance that suppresses or inhibits the expression of MERUMUC and a pharmaceutically acceptable carrier;
(21) the pharmaceutical composition of (20), wherein the substance inhibits the transcription of a gene encoding MERMUC to mRNA;
(22) the pharmaceutical composition of (20), wherein the substance inhibits the translation of an mRNA encoding MERMUC to protein;
(23) the pharmaceutical composition of (20), wherein said HGF is an endogenous HGF of an organism;
(24) the pharmaceutical composition of (20), wherein said HGF is an HGF drug that can exogenously provide HGF to an organism;
(25) the pharmaceutical composition of (24), wherein the HGF drug is a protein drug comprising an HGF protein;
(26) the pharmaceutical composition of (24), wherein the HGF drug is a DNA drug comprising DNA encoding an HGF protein;
(27) the pharmaceutical composition of (17) or (20), which is used for preventing or treating fibrosis;
(28) the pharmaceutical composition of (27), wherein the fibrosis is hepatic, renal or pulmonary fibrosis;
(29) the pharmaceutical composition of (17) or (20), which is used for preventing or treating hepatic cirrhosis, hepatic fibrosis or hepatitis;
(30) the pharmaceutical composition of (17) or (20), wherein hepatitis is used for preventing or treating fulminant hepatitis, acute hepatitis or viral hepatitis;
(31) the pharmaceutical composition of (17) or (20), which is used for preventing or treating chronic renal failure;
(32) the pharmaceutical composition of (17) or (20), which is used for preventing or treating arteriosclerosis obliterans;
(33) a polypeptide comprising a partial amino acid sequence of the full-length amino acid sequence of MERMUC, and having activity to bind c-Met;
(34) the polypeptide of (33), wherein said partial amino acid sequence corresponds to whole or a portion of a cytoplasmic region of MERMUC;
(35) the polypeptide of (35), wherein said whole or a portion of a cytoplasmic region comprises whole or a portion of a leucine repeat region in the C-terminal region of MERMUC;
(36) the polypeptide of (33), wherein said MERMUC is human MERMUC;
(37) the polypeptide of (33), which comprises the amino acid sequence of SEQ ID NO: 15;
(38) a polypeptide comprising a partial amino acid sequence of the full-length amino acid sequence of c-Met, and having activity to bind MERMUC;
(39) the polypeptide of (38), wherein said partial amino acid sequence corresponds to whole or a portion of a cytoplasmic region of the c-Met β chain;
(40) the polypeptide of (39), wherein said whole or a portion of a cytoplasmic region comprises whole or a portion of a C-terminal region of c-Met comprising one or more tyrosine residues that undergo self-phosphorylation;
(41) the polypeptide of (38), wherein said c-Met is human c-Met;
(42) the polypeptide of (38), which comprises the amino acid sequence of SEQ ID NO: 5;
(43) a polypeptide comprising a partial amino acid sequence of the full-length amino acid sequence of MERMUC, and comprising the activity to bind MERMUC;
(44) the polypeptide of (43), wherein said partial amino acid sequence corresponds to whole or a portion of a cytoplasmic region of MERMUC;
(45) the polypeptide of (33), wherein said MERMUC is human MERMUC;
(46) a fusion polypeptide represented by the general formula W—X—Y (wherein, W is absent, or the full-length amino acid sequence or a portion of a protein other than MERMUC; X is the full-length amino acid sequence of MERMUC or a portion thereof; and Y is the full-length amino acid sequence or a portion of a protein other than MERMUC);
(47) the fusion polypeptide of (46), wherein the protein Y is β-galΔα or β-galΔω;
(48) the fusion polypeptide of (47), wherein said β-galΔα comprises the amino acid sequence of SEQ ID NO: 16;
(49) the fusion polypeptide of (47), wherein said β-galΔω comprises the amino acid sequence of SEQ ID NO: 17;
(50) the fusion polypeptide of (46), wherein said portion of the full-length amino acid sequence of MERMUC has activity to bind c-Met;
(51) the fusion polypeptide of (46), wherein said portion of the full-length amino acid sequence of MERMUC comprises whole or a portion of a cytoplasmic region of MERMUC;
(52) the fusion polypeptide of (51), wherein said portion of the full-length amino acid sequence of MERMUC comprises whole or a portion of a leucine repeat region of the C-terminal region of MERMUC;
(53) the fusion polypeptide of (46), wherein said MERMUC is human MERMUC;
(54) the polypeptide of (46), wherein said X comprises the amino acid sequence of SEQ ID NO: 15;
(55) the polypeptide of (46), wherein the protein W comprises the extracellular region of Fas;
(56) a fusion polypeptide represented by the general formula Z-Y (wherein Z is the full-length amino acid sequence of c-Met or a portion thereof; and Y is the full-length amino acid sequence or a portion of a protein other than c-Met);
(57) the fusion polypeptide of (52), wherein said protein is β-galΔα or β-galΔω;
(58) the fusion polypeptide of (57), wherein said βgalΔα comprises the amino acid sequence of SEQ ID NO: 16;
(59) the fusion polypeptide of (57), wherein said β-galΔω comprises the amino acid sequence of SEQ ID NO: 17;
(60) the fusion polypeptide of (56), wherein said portion of the full-length amino acid sequence of c-Met has activity to bind MERMUC;
(61) the fusion polypeptide of (56), wherein said portion of the full-length amino acid sequence of c-Met comprises whole or a portion of a cytoplasmic region of the c-Met β chain;
(62) the fusion polypeptide of (56), wherein said portion of the full-length amino acid sequence of c-Met comprises whole or a portion of a C-terminal region of c-Met comprising one or more tyrosine residues that undergo self-phosphorylation;
(63) the fusion polypeptide of (56), wherein said c-Met is human c-Met;
(64) the fusion polypeptide of (56), wherein said Z comprises the amino acid sequence of SEQ ID NO: 5;
(65) a method of testing a substance for the presence or extent of activity to inhibit the binding between c-Met and MERMUC, which comprises the step of comparing the presence or extent of the binding between c-Met and MERMUC in the presence of a test substance to the extent of the binding between c-Met and MERMUC in the absence of the test substance;
(66) a method of identifying a substance that has activity to inhibit the binding between c-Met and MERMUC, which comprises the step of comparing the presence or extent of the binding between c-Met and MERMUC in the presence of a test substance to the extent of the binding between c-Met and MERMUC in the absence of the test substance;
(67) a method of testing for the presence or extent of activity of a substance to inhibit the binding between c-Met and MERMUC, which comprises the steps of:
(a) preparing a cell that is designed to coexpress c-Met and MERMUC, and allow detection of the binding between said c-Met and MERMUC;
(b) culturing the cell obtained in step (a) in the presence of HGF while in the absence of test substance, and then measuring the extent of the binding between c-Met and MERMUC in the cell;
(c) culturing the cell obtained in step (b) in the presence of HGF and a test substance, and then measuring the extent of the binding between c-Met and MERMUC in the cell; and
(d) comparing the measurement result of step (b) with that of step (c);
(68) the method of (67), wherein said MERMUC is the fusion polypeptide of any one of (46) to (54);
(69) the method of (67), wherein said MERMUC is the fusion polypeptide of any one of (47) to (49);
(70) the method of (67), wherein said c-Met is the fusion polypeptide of any one of (56) to (64);
(71) the method of (67), wherein said c-Met is the fusion polypeptide of any one of (57) to (59);
(72) a method of identifying a substance that has activity to inhibit the binding between c-Met and MERMUC, which comprises the steps of:
(a) preparing a cell that is designed to coexpress c-Met and MERMUC, and allow detection of the binding between said c-Met and MERMUC bind;
(b) culturing the cell obtained in step (a) in the presence of HGF while in the absence of test substance, and then measuring the extent of the binding between c-Met and MERMUC in the cell;
(c) culturing the cell obtained in step (b) in the presence of HGF and a test substance, and then measuring the extent of the binding between c-Met and MERMUC in the cell; and
(d) comparing the measurement result of step (b) with that of step (c);
(73) the method of (72), wherein said MERMUC is the fusion polypeptide of any one of (46) to (54);
(74) the method of (72), wherein said MERMUC is the fusion polypeptide of any one of (47) to (49);
(75) the method of (72), wherein said c-Met is the fusion polypeptide of any one of (56) to (64);
(76) the method of (72), wherein said c-Met is the fusion polypeptide of any one of (57) to (59);
(77) a method of testing a substance for the presence or extent of activity to inhibit multimerization of MERMUC, which comprises the step of comparing the presence or extent of multimerization of MERMUC in the presence of a test substance to the extent of multimerization of MERMUC in the absence of the test substance; and
(78) a method for identifying a substance having activity to inhibit multimerization of MERMUC, which comprises the step of comparing the presence or extent of multimerization of MERMUC in the presence of a test compound to the extent of multimerization of MERMUC in the absence of the test substance.

The present invention is specifically described by defining the meaning of the terms used in this invention and methods of producing the substances, as follows.

In the present invention, the term “mammal” refers to humans, cattle, goats, rabbits, mice, rats, hamsters, guinea pigs and such, preferably humans, cattle, rats, mice or hamsters, and particularly preferably humans.

Herein, the term “c-Met” refers to the c-Met of the above-mentioned mammals, preferably human, mouse or rat c-Met, and particularly preferred is human c-Met. c-Met is a receptor of hepatocyte growth factor (HGF) defined below, and c-Met has the structure and biological function as previously reported.

c-Met is a heterodimer of disulfide-bonded α chain (molecular weight: approximately 50 kDa) consisting of only the extracellular region, and β chain (molecular weight: approximately 145 kDa) which is a transmembrane protein.

Human c-Met is a preferred embodiment of the present invention, and isomers of c-Met (isomer 1, isomer 2, etc.) exist for human c-Met. The human c-Met of this invention includes all of these isomers. Isomer 1 and isomer 2 are considered splice variants to each other.

The primary structures (the precursor of the heterodimer) of human c-Met reported as isomer 1 and isomer 2 comprise 1390 amino acid residues (SEQ ID NO: 2; GenBank Accession Nos. P08581, TVHUME and CAB56793; the corresponding cDNA nucleotide sequence is shown in SEQ ID NO: 1) and 1408 amino acid residues (GenBank Accession No. 4557747; corresponding), respectively.

The only difference between isomer 1 and isomer 2 is the 18 amino acid residues present between amino acids 754 (Ile) and 755 (Ser) of isomer 1, and the rest of the amino acid sequence is identical.

Hereinafter, c-Met will be described and defined using isomer 1 as an example; however, it should be construed that isomer 2 which has a similar structure is also encompassed by the present invention.

Human c-Met consists of signal peptide (amino acids 1-24 of SEQ ID NO: 2), α chain (amino acids 25-303 of SEQ ID NO: 2) and β chain (amino acids 308-1390 of SEQ ID NO: 2; identical to the full-length sequence of SEQ ID NO: 4; SEQ ID NO: 3 is the corresponding cDNA sequence) (Proc. Natl. Acad. Sci. USA., 1987, Vol.84, No.8, p.6379-6383; Oncogene, 1987, Vol.1, No.2, p.229-233; Oncogene, 1993, Vol.8, No.12, p.3403-3410; Nature, 1985, Vol.318, No.6044, p.385-388; Science, 1991, Vol.251, No.4995, p.802-804; J. Biol. Chem., 1991, Vol.266, No.29, p.19558-19564; Nature Genetics, 1997, Vol.16, No.1, p.68-73; Mol. Cell. Biol., 1987, Vol.7, No.2, p.921-924; J. Biol. Chem., 1994, Vol.269, No.17, p.12852-12857).

The structural and biological characteristics of both mouse and rat c-Met are similar to human c-Met ([mouse c-Met] GenBank Accession Nos. S01254, NP 032617, P16056, and CAA68680; Oncogene, 1988, Vol.2, No.6, p.593-599; Gene, 1989, Vol.85, No.1, p.67-74; J. Cell Biol., 1993, Vol.121, No.1, p.145-154. [rat] GenBank Accession Nos. NP 113705 and AAB19189; Am. J. Physiol.1996, Vol.271, No.3 Pt 2, p. F679-F688).

The β chain of c-Met (in humans, amino acids 308-1390 of SEQ ID NO: 2; same as the full-length sequence of SEQ ID NO: 4) is composed of an extracellular region (in humans, amino acids 308-932 of SEQ ID NO: 2; same as amino acids 1-625 of SEQ ID NO: 4), transmembrane region (in humans, amino acids 933-955 of SEQ ID NO: 2; same as amino acids 626-648 of SEQ ID NO: 4) and cytoplasmic region (in humans, amino acids 956-1390 of SEQ ID NO: 2; same as amino acids 649-1083 of SEQ ID NO: 4). The cytoplasmic region comprises a tyrosine kinase domain (in humans, amino acids 1078-1345 of SEQ ID NO: 2; same as amino acids 771-1038 of SEQ ID NO: 4). When HGF binds to the receptor c-Met, dimerization of the receptor takes place, and subsequently induces self-phosphorylation of tyrosine residues that exist in the cytoplasmic region (in humans, the tyrosine residues of amino acids 1349, 1356 and 1365 of SEQ ID NO: 2; same as amino acids 1042, 1049 and 1058 of SEQ ID NO: 4).

Furthermore, a site comprising 3 phosphorylated tyrosine residues (in humans, amino acids 1349, 1356 and 1365 of SEQ ID NO: 2; same as amino acids 1042, 1049 and 1058 of SEQ ID NO: 4) called multifunctional docking site exists on the C-terminal side of the c-Met β chain. The binding of various signal transduction molecules comprising an SH2 domain, such as PI3 kinase, PLC-γ, SHC, Grb-1, Grb-2 and pp60^{c-SRC}, to this site, in particular, causes transmission of signals responsible in various biological activities of HGF (Cell, 1994, Vol.77, p.261-271; Experimental Medicine, 1997, Vol.15, p.1033-1039).

The phrase “cytoplasmic region of the c-Met β chain” in this invention preferably refers to the cytoplasmic region of the c-Met β chain of human, mouse or rat, and particularly preferred is the cytoplasmic region of the human c-Met β chain (amino acids 956-1390 of SEQ ID NO: 2; same as amino acids 649-1083 of SEQ ID NO: 4).

The phrase “a portion of the cytoplasmic region of the c-Met β chain” in this invention refers to any arbitrary portion of the above-mentioned cytoplasmic region. However, the C-terminal region of the β chain is preferred, and a portion binding to the C-terminal region MERMUC, defined infra, which comprises one or more tyrosine residues that undergo self-phosphorylation (in humans, amino acids 1349, 1356 and 1365 of SEQ ID NO: 2; the same as amino acids 1042, 1049 and 1058 of SEQ ID NO: 4) is particularly preferred. More specifically, it refers to a region comprising the shortest amino acid sequence required in the binding with MERMUC (SEQ ID NO: 5), which sequence is indicated in the Examples described below. However, this shortest amino acid sequence refers to the sequence based only on limited experimental results disclosed in the Examples described below. Thus, the term “smallest unit” in the present invention also refers to shorter amino acid sequences than this sequence of SEQ ID NO: 5.

The human c-Met of this invention includes isomers of human c-Met having the primary structures described above (one of the isomers, isomer 2 comprises 1408 amino acids; Proc. Natl. Acad. Sci. USA., 1987, Vol.84, No.18, p.6379-6383; Nature, 1985, Vol.318, No.6044, p.385-388; Mol. Cell. Biol., 1987, Vol.7, No.2, p.921-924; Science, 1991, Vol.251, No.4995, p.802-804; Cytogenet. Cell Genet., 1992, Vol.60, No.2, p.114-116; Cell. Mol. Biol. Res., 1994, Vol.40, No.4, p.337-350; Cell Mol. Biol. Res., 1994, Vol.40, No.7-8, p.7-7). The primary structure of the two isomers differ only in a portion of the amino acid sequence within the extracellular region, and the amino acid sequences from the C-terminal side of the extracellular region through the cytoplasmic region to the C-terminus are identical. This means that the structure of the region required for the binding to MERMUC, demonstrated in the following Examples, is conserved in both forms of the human c-Met β chains.

In the present invention, the term “HGF” refers to a mammal-derived hepatocyte growth factor (HGF), which is a ligand of c-Met defined above. Preferred HGF includes those from human, mouse and rat, and human HGF is particularly preferred.

HGF is synthesized as single-stranded inactive prepro form. Then, it is processed by a serine protease called HGF converting enzyme or HGF activator to form the active heterodimer consisting of an α chain (molecular weight of approximately 69 kDa) and a β chain (molecular weight of approximately 34 kDa). This active heterodimer exhibits various biological activities of HGF through the binding to an HGF receptor (see supra).

The prepro form of human HGF comprises 728 amino acids (amino acid sequence: SEQ ID NO: 7; corresponding cDNA sequence: SEQ ID NO: 6) (GenBank Accession Nos. JH0579, P14210, XP 052257, XP 052258, XP 052260, AAA52648, BAA14348, AAA52650 and AAA64239; Biochem. Biophys. Res. Commun., 1989, Vol.163 No.2, p.967-973; Nature, 1989, Vol.342, No.6248, p.440-443; Biochem. Biophys. Res. Commun., 1990, Vol.172, No.1, p.321-327; J. Cell Biol., 1990, Vol.111, No.5, Pt.1, p.2097-2108; Proc. Natl. Acad. Sci. USA., 1991, Vol.88, No.2, p.415-419; Biochem. Biophys. Res. Commun., 1991, Vol.175, No.2, p.660-667; Eur. J. Biochem., 1991, Vol.197, No.1, p.15-22; Gene, 1991, Vol.102, No.2, p.213-219; Proc. Natl. Acad. Sci. USA., 1991, Vol.88, No.16, p.7001-7005; Biochem. Biophys. Res. Commun., 1991, Vol.180, No.2, p.1151-1158; Proc. Natl. Acad. Sci. USA., 1992, Vol.89, No.23, p.11574-11578; Biochem. Biophys. Res. Commun., 1992, Vol.189, No.3, p.1329-1335; EMBO J., 1992, Vol.11, No.7, p.2503-2510; Structure, 1998, Vol.6, No.1, p.109-116; Structure, 1998, Vol.6, No.11, p.1383-1393).

Similar to human HGF, the prepro forms of rat HGF and mouse HGF are also composed of 728 amino acids, and have structural and biological characteristics as reported previously ([rat] GenBank Accession Nos. NP058713, P17945, A35644, BAA14133and CAA38266; Proc. Natl. Acad. Sci. USA., 1990, Vol.87, No.8, p.3200-3204; Eur. J. Biochem., 1990, Vol.193, No.2, p.375-381. [mouse] GenBank Accession Nos. A60185, Q08048, BAA01064 and CAA58865; Nature, 1990, Vol.346, No.6281, p.228; Proc. Natl. Acad. Sci. USA., 1990, Vol.195, No.1, p.34-43; Biochem. J., 1991, Vol.278, Pt.1, p.35-41; Biochem. Biophys. Acta, 1993, Vol.1216, No.2, p.299-303, 1993; Biochem. Biophys. Res. Commun., 1994, Vol.199, No.2, p.772-779;J. Biol. Chem., 1995, Vol.270, No.2, p.830-836; Cell Adhes. Commun., 1993, Vol.1, No.2, p.101-111).

In the present invention, the term “MERMUC” refers to a novel mammalian mucin-like molecule described in International Application No. WO 01/05803. Herein, this mucin-like molecule is called c-Met Regulatory Mucin (MERMUC). A preferred embodiment of the present invention is human, mouse or rat MERMUC, and human MERMUC is particularly preferred.

The present inventors made every effort to study MERMUC, and identified that MERMUC has at least the structural characteristics described below, and in the Examples:
(1) the primary structure of mammalian MERMUC comprises extracellular region, a transmembrane region and cytoplasmic region;
(2) a mucin-specific domain known as a potential O-linked glycosylation site exists in the extracellular region. This domain comprises a repeat structure of particular amino acid sequence rich in serine/threonine residues that can be glycosylated (in human MERMUC, the 19 amino acid residues of SEQ ID NO: 8);
(3) the number of repeating units in the mucin-specific domain is variable, and polymorphism exists in MERMUC depending on the number of repeating units;
(4) a leucine repeat region exists in the C-terminal region, also the terminus of the cytoplasmic region, of MERMUC. This repeat structure is highly conserved among humans, mice and rats; and
(5) MERMUC may exist as a monomer of a molecule having the above-mentioned primary structure, or as a multimer (oligomer, such as dimer or trimer) formed by association or binding of two or more of the molecules.

Therefore, in the present invention, MERMUC refers to a molecule having structural characteristics as discussed above (including the characteristics previously reported in International Application No. WO 01/05803). The present invention encompasses all forms of any polymorphs, monomers and multimers (dimers, oligomers, etc.).

The primary structure of human MERMUC of this invention in which such polymorphs exist includes, for example, SEQ ID NO: 10 (extracellular region: amino acids 1-199, transmembrane region: amino acids 200 to 220 or 221, and cytoplasmic region: from 221 or 222 to 503; cDNA sequence: SEQ ID NO: 9). As mentioned above, while polymorphisms exist for human MERMUC that differ in the repeat structures of the mucin-specific domain of the extracellular region, the structures of the cytoplasmic region are the same.

The primary structure of mouse MERMUC is exemplified by SEQ ID NO: 12 (cDNA sequence: SEQ ID NO: 11). An example of the primary structure of rat MERMUC (lacking the N-terminus) is SEQ ID NO: 14 (cDNA sequence: SEQ ID NO: 13).

The phrase “C-terminal region of MERMUC” as employed herein refers to the amino acid sequence of the carboxyl terminus of the cytoplasmic region of MERMUC, and the region comprising a leucine repeat structure is preferred. In the interest of human MERMUC, the region comprising 53 amino acids of the C-terminus (SEQ ID NO: 15) can be mentioned as a specific example.

The terms “β-gal”, “β-galΔα” and “β-galΔω” of this invention each have the meaning defined below:

The term “β-gal” is an abbreviation of β-galactosidase (lactase), and refers to an enzyme that degrades and synthesizes lactose (glucose-β-D-galactoside). This enzyme is widely distributed from bacteria to higher animals and plants, and the structure and optimum pH differ depending on the species.

β-gal of this invention encompasses β-gal of all kind of species; however, β-gal of E. coli is particularly preferred.

The structural gene encoding β-gal of E. coli is called lacZ. The primary structure of the precursor of E. coli β-gal comprises 1024 amino acids (GenBank Accession Nos. P00722 and GBEC; J. Biol. Chem., 1978, Vol.253, No.15, p.5521-5525; Bioorg. Khim., 1980, Vol.6, p.1735-1736; Nature, 1980, Vol.285, No.5759, p.38-41; EMBO J., 1983, Vol.2, No.4, p.593-597; J. Mol. Biol., 1989, Vol.208, No.1, p.23-43; Gene, 1992, Vol.122, No.1, p.231-232; Nature, 1994, Vol.369, No.6483, p.761-766; Science, 1997, Vol.277, No.5331, p.1453-1474).

The biological activity of β-gal is expressed by a homotetramer formed via the association of 4 monomeric molecules having this primary structure (according to a three-dimensional structural analysis of E. coli β-gal, the primary structure of each monomer molecule has been reported to consist of 1021 amino acids lacking 3 amino acids of the N-terminus; GenBank Accession Nos. 13399708; Nature, 1994, Vol.369, No.6483, p.761-766; Protein Sci., 1999, Vol.8, No.1, p.122-136; Protein Sci., 2000, Vol.9, No.9, p.1685-1699).

On the other hand, in the interest of β-gal of E. coli, three mutants, β-galΔα, β-galΔμ and β-galΔω have been reported in a type of E. coli strain (A denotes deletion; Proc. Natl. Acad. Sci. USA., 1996, Vol.93, p.12423-12427; Proc. Natl. Acad. Sci. USA., 1997, Vol.94, p.8405-8410). β-galΔα lacks a portion (amino acids 12-42 of GenBank Accession No. P00722) of the N-terminal sequence which is the active site of wild-type β-gal. β-galΔμ lacks a portion (amino acids 50-602 of GenBank Accession No. P00722) of the central sequence of wild-type β-gal. Furthermore, β-galΔω lacks a portion (amino acids 790-1024 of GenBank Accession No. P00722) of the C-terminal sequence, a site for forming homotetramer in the wild-type β-gal monomers.

The term “β-galΔα” of the present invention refers to any mutant (including naturally occurring and artificially prepared mutants) lacking a portion of the N-terminal sequence which is the active site of wild-type β-gal (particularly preferred, β-gal of E. coli), and the previously reported mutant β-galΔα (SEQ ID NO: 16) is particularly preferred.

The term “β-galΔω” of the present invention refers to any mutant (including naturally occurring and artificially prepared mutants) lacking a portion of the C-terminal sequence which is the site for forming homotetramer in the wild-type P-gal monomer molecules, and the previously reported mutant β-galΔω (SEQ ID NO: 17) is particularly preferred.

“c-Met” in the definition of “fusion polypeptide represented by general formula W—X—Y”, and “MERMUC” in the definition of “fusion polypeptide represented by general formula Z-Y” herein have the same meaning as the above-mentioned c-Met and MERMUC, respectively.

Y in the fusion polypeptide represented by the general formula W—X—Y denotes the full-length amino acid sequence of the entire polypeptide or a portion of a protein other than MERMUC. Herein, the term “protein other than” encompasses any protein; however, preferably, it refers to a protein having a characteristic that enables measurement of the presence or degree of the binding between c-Met and MERMUC, and particularly preferred examples include β-galΔα and β-galΔω mentioned above.

W in the fusion polypeptide, represented by the general formula W—X—Y, denotes the full-length amino acid sequence of the entire polypeptide or a portion of a protein other than MERMUC.

Herein, the term “protein other than” encompasses any protein. However, preferably, it refers to a protein having a characteristic that enables measurement of the presence or degree of multimerization of MERMUC, and particularly preferred examples include mouse, rat and human Fas (<mouse> GenBank Accession No. P25446 and NP032013; <human> GenBank Accession No. P25445 and NP 000034).

Yin the fusion peptide, represented by the general formula Z-Y, denotes the full-length amino acid sequence or a portion of the entire polypeptide of a protein other than c-Met. Herein, the term “protein other than” encompasses any protein; however, preferably, it refers to a protein having a characteristic that enables measurement of the presence or degree of the binding between c-Met and MERMUC, and particularly preferred examples include β-galΔα and β-galΔω mentioned above.

In the polypeptide or fusion polypeptide of this invention, a plurality of amino acids, preferably 1 to 10, and particularly preferably 1 to 5 amino acids within the amino acid sequence may be replaced, deleted and/or modified, or a plurality of amino acids, preferably 1 to 10, particularly preferably 1 to 5 amino acids may be added to the amino acid sequence as long as it has the desired property or accomplishes the desired objective.

The phrase “HGF drugs” of this invention refers to a drug for supplying hepatocyte growth factor (HGF) defined above, particularly preferably human HGF to an organism (particularly preferably human) and includes “protein drugs” and “DNA drugs”.

A protein drug is a pharmaceutical agent comprising an HGF protein (human HGF is particularly preferred; it may be purified HGF from a cell culture or a recombinant HGF) and a pharmaceutically acceptable carrier.

The DNA drug is a pharmaceutical agent comprising a “DNA structure” carrying a gene (cDNA or genomic DNA) encoding an HGF (human HGF is particularly preferred) and a pharmaceutically acceptable carrier, yet used for what is called gene therapy.

Herein, the “DNA structure” refers to a structure wherein a gene encoding HGF is inserted in an expressible manner in a cell (particularly preferred muscle cells) of a host (specifically, the organism to which this DNA drug is administered). For example, it includes plasmid vectors and virus vectors carrying the HGF gene. In the present invention, vectors include any arbitrary plasmid vectors and virus vectors used in the field of gene therapy.

The “substance” or “test substance” of the present invention refers to natural substances or any artificially prepared substances. Specific examples encompass chemically synthesized substances (low-molecular weight compounds and such), polypeptides (natural form, recombinant form, cultured form, etc.), antibodies, DNAs and RNAs.

“Substances having activity to inhibit the binding between c-Met and MERMUC” of this invention includes any substance as long as it has activity to inhibit the binding between c-Met and MERMUC. Therefore, in the present invention, there is no limitation on the mechanism (inhibition mechanism) by which the substance exhibits the inhibitory activity on the binding between c-Met and MERMUC. Specifically, such substance includes those functioning through the following inhibition mechanism. However, the invention should not be construed as being limited to these examples:
(1) a substance that inhibits the binding between c-Met and MERMUC via direct binding to the MERMUC binding site or its vicinity (including three dimensional space) on c-Met;
(2) a substance that inhibits the binding between c-Met and MERMUC via direct binding to the c-Met binding site or its vicinity (including three dimensional space) on MERMUC;
(3) a substance that inhibits the binding between c-Met and MERMUC as a result of changing the three-dimensional structure of MERMUC or inhibiting the multimerization of MERMUC via the binding to an arbitrary region (e.g., the extracellular region) of MERMUC (for example, MERMUC-binding antibodies); and
(4) a substance that inhibits the binding between c-Met and MERMUC because of dissociating the bond of bound c-Met and MERMUC.

“A substance having activity to inhibit multimerization of MERMUC” of this invention includes any substance as long as it has activity to inhibit multimerization due to self-association and bonding of MERMUC molecules. Therefore, in the present invention, the mechanism (inhibition mechanism) by which the substance exhibits the activity of inhibiting multimerization of MERMUC molecules is not limited. Specifically, such substances include those functioning through the following inhibition mechanism. However, the present invention should not be construed as being limited to these examples:
(1) a substance that inhibits self-association (multimerization) of MERMUC via direct binding to the site at which 2 or more MERMUC molecules bind for self-association (specifically, the cytoplasmic region) or to its vicinity (including three dimensional space); and
(2) a substance that inhibits self-association (multimerization) of 2 or more MERMUC molecules as a result of changing the three-dimensional structure of MERMUC or by inhibiting multimerization of MERMUC via the binding to an arbitrary region (e.g., the extracellular region) of MERMUC (for example, MERMUC-binding antibodies).

“Substances that suppress or inhibit the expression of MERMUC” of this invention include any substance as long as it has the activity of suppressing or inhibiting the expression of the MERMUC molecule on the cell membrane. Therefore, in the present invention, there are no limitations on the mechanism (inhibition mechanism) by which the substance exhibits this inhibitory activity. Specifically, such substances include those functioning through the following inhibition mechanism. However, the present invention should not be construed as being limited to these examples:
(1) a substance that suppresses or inhibits the transcription of a gene encoding MERMUC to mRNA; and
(2) a substance that inhibits or suppresses the translation of an mRNA encoding MERMUC to protein.

The above-mentioned (1) encompasses not only the following antisense DNA, but also substances having activity to modulate the activity of a promoter and/or a transcription factor that modulates the transcription of a gene encoding MERMUC to mRNA.

Each of the chemically synthesized substances (low-molecular weight compounds and such), polypeptides, antibodies, DNA and RNA encompassed by the “substances” composing the “pharmaceutical composition” of this invention is defined as follows.

Examples of the “antibodies” include MERMUC (particularly preferred, human MERMUC)-binding polyclonal antibodies, monoclonal antibodies and portions of the monoclonal antibodies. Monoclonal antibodies and portions thereof are preferred. The monoclonal antibodies include in addition to non-human mammal-derived monoclonal antibodies, recombinant chimeric monoclonal antibodies (Experimental Medicine (supplementary volume), 1988, Vol.1.6, No. 10; Examined Published Japanese Patent Application (JP-B) No. Hei 3-73280; etc.), recombinant humanized monoclonal antibodies and human monoclonal antibodies (Nature Genetics, 1994, Vol.7, p.13-21; Nature Genetics, 1997, Vol.15, p.146-156; Published Japanese Translation of International Publications Nos. Hei 4-504365 and Hei 7-509137; Nikkei Science, June 1995, p.40-50; International Application No. WO 94/25585; Nature, 1994, Vol.368, p.856-859; Published Japanese Translation of International Publication No. Hei 6-500233; Nikkei Science, April 1997, p.78-84; etc.).

The term “polypeptide” (herein, it refers to polypeptides that are components of a pharmaceutical composition of this invention, and not to polypeptides themselves as another invention) encompasses oligopeptides, fusion polypeptides and chemically modified forms thereof. Examples of oligopeptides are peptides comprising 5 to 30 amino acids, preferably 5 to 20 amino acids. A chemical modification can be designed depending on various purposes, for example, to increase half-life in blood in the case of administering in vivo, or to increase tolerance against degradation or increase absorption in the digestive tract in oral administrations.

The term “DNA” means “DNA comprising a partial nucleotide sequence of an antisense DNA or a chemically modified DNA thereof”, which DNA is designed based on the nucleotide sequence of the DNA (including cDNA and genomic DNA) encoding MERMUC, yet useful as an antisense DNA pharmaceutical. Specifically, the antisense DNA can inhibit the transcription of gene encoding MERMUC into mRNA or the translation of the mRNA into a protein by hybridizing to the gene or RNA encoding MERMUC.

The term “RNA” means “RNA comprising a partial nucleotide sequence of an antisense RNA or a chemically modified RNA thereof”, which RNA is designed based on the nucleotide sequence of the RNA encoding the MERMUC, yet useful as an antisense RNA pharmaceutical. The antisense RNA can inhibit the transcription of the gene encoding MERMUC into mRNA or the translation of the mRNA into a protein by hybridizing to the gene or RNA encoding MERMUC.

The expression “partial nucleotide sequence” as employed herein, refers to a partial nucleotide sequence comprising an arbitrary number of nucleotides of an arbitrary region. A partial nucleotide sequence includes 5 to 100 consecutive nucleotides, preferably 5 to 70 consecutive nucleotides, more preferably 5 to 50 consecutive nucleotides, and even more preferably 5 to 30 consecutive nucleotides.

When the DNA or RNA is used as an antisense pharmaceutical, the DNA or RNA sequence can be chemically modified in part in order to extend the half-life (stability) in blood when the DNA or RNA is administered to patients, to increase the intracytoplasmic-membrane permeability of the DNA or RNA, or to increase the degradation resistance or the absorption of orally administered DNA or RNA in the digestive organs. Chemical modifications include, for example, the modification of a phosphate bond, a ribose, a nucleotide, the sugar moiety, and the 3∝ end and/or the 5′ end in the structure of an oligonucleotide.

Modifications of phosphate bonds include, for example, the conversion of one or more bonds to phosphodiester bonds (D-oligo), phosphorothioate bonds, phosphorodithioate bonds (S-oligo), methyl phosphonate (MP-oligo) bonds, phosphoroamidate bonds, non-phosphate bonds or methyl phosphonothioate bonds, or combinations thereof. Modification of a ribose includes, for example, the conversion to 2′-fluororibose or 2′-O-methylribose. Modification of a nucleotide includes, for example, the conversion to 5-propynyluracil or 2-aminoadenine.

The phrase “chemically synthesized substance” or “low-molecular weight compound” refers to any compound except the above-mentioned antibodies, polypeptides, DNA and RNA, and examples include compounds having a molecular weight of approximately 100 to approximately 1000 or less, preferably approximately 100 to approximately 800, and more preferably approximately 100 to approximately 600.

The polypeptides, portions of the polypeptides (fragment) and fusion polypeptides mentioned above can be produced not only by recombinant DNA technology as mentioned below, but also by methods well known in the art, such as chemical synthetic methods or cell culture methods, or modified methods thereof.

The “antibodies that bind to MERMUC” of this invention include natural antibodies obtained by immunizing mammals such as mice, rats, hamsters, guinea pigs and rabbits, with an antigen, chimeric antibodies and humanized antibodies (CDR-grafted antibody) that may be produced using gene recombination techniques, and human antibodies that may be produced using human antibody-producing transgenic animals and such. The natural antibodies may be produced using cells (natural cells, established cell lines, tumor cells, etc.) that express MERMUC (particularly preferably human MERMUC), transformants produced using gene recombination techniques so that MERMUC (particularly preferably human MERMUC) is overexpressed on the cell surface, polypeptides (particularly preferably polypeptides that compose the extracellular region) composing MERMUC (particularly preferably human MERMUC), or fusion polypeptides comprising all or a portion of the extracellular region of this MERMUC and all or a portion of another protein (human IgFc, glutathione-S-transferase, His tag, β-galactosidase, etc.) as antigens.

Monoclonal antibodies having any one isotype of IgG, IgM, IgA, IgD or IgE. IgG or IgM are preferred.

A polyclonal antibody (antisera) or monoclonal antibody can be produced by known methods. Specifically, a mammal, preferably, a mouse, rat, hamster, guinea pig, rabbit, cat, dog, pig, goat, horse or cow, or more preferably, a mouse, rat, hamster, guinea pig or rabbit is immunized, for example, with an antigen mentioned above with Freund's adjuvant, if necessary.

A polyclonal antibody can be obtained from the serum obtained from the animal so immunized. On the other hand, monoclonal antibodies are produced as follows. Hybridomas are prepared from the antibody-producing cells obtained from the animal so immunized and myeloma cells that are not capable of producing autoantibodies. The hybridomas are cloned, and clones producing the monoclonal antibodies showing a specific affinity to the antigen used for immunizing the mammal are screened.

Specifically, a monoclonal antibody can be produced as follows. Immunizations are performed by injecting or implanting once or several times an antigen mentioned above as an immunogen, if necessary, with Freund's adjuvant, subcutaneously, intramuscularly, intravenously, through the footpad, or intraperitoneally into a non-human mammal, specifically a mouse, rat, hamster, guinea pig or rabbit, preferably a mouse, rat or hamster (including a transgenic animal generated so as to produce antibodies derived from another animal, such as a transgenic mouse producing human antibody mentioned below). Usually, immunizations are performed once to four times every one to fourteen days after the first immunization. Antibody-producing cells are obtained from the mammal so immunized in about one to five days after the last immunization. The frequency and interval of immunizations can be appropriately arranged depending on, for example, the property of the immunogen used.

Hybridomas that secrete a monoclonal antibody can be prepared by the method of Köhler and Milstein (Nature, 1975, Vol.256, p.495-497), or by a modified method thereof. Specifically, hybridomas are prepared by fusing antibody-producing cells contained in a spleen, lymph node, bone marrow or tonsil, preferably a spleen, obtained from a non-human mammal immunized as mentioned above with myelomas without an autoantibody-producing ability, which are derived from, preferably, a mammal such as a mouse, rat, guinea pig, hamster, rabbit or human, or more preferably, a mouse, rat or human.

For example, a mouse-derived myeloma P3/X63-AG8.653 (653), P3/NSI/1-Ag4-1 (NS-1) , P3/X63-Ag8.U1 (P3U1), SP2/0-Ag14 (Sp2/0, Sp2), PAI, F0 or BW5147, rat-derived myeloma 210RCY3-Ag.2.3., or human-derived myeloma U-266AR1, GM1500-6TG-A1-2, UC729-6, CEM-AGR, DlR11 or CEM-T15 can be used as a myeloma for cell fusion.

Hybridomas producing monoclonal antibodies can be screened by cultivating hybridomas, for example, in microtiter plates and by measuring the reactivity of the culture supernatant in wells in which hybridoma growth is observed, to the immunogen used for the immunization mentioned above, for example, by an enzyme immunoassay such as RIA and ELISA.

Monoclonal antibodies can be produced from hybridomas by cultivating the hybridomas in vitro or in vivo such as in the ascites fluid of a mouse, rat, guinea pig, hamster or rabbit, preferably a mouse or rat, more preferably mouse, and isolating the antibodies from the resulting culture supernatant or ascites fluid of a mammal.

In vitro cultivation of hybridomas can be performed depending on, e.g., the property of cells to be cultured, the object of the study and various conditions of the culture method, using known nutrient media or any nutrient media derived from known basal media for growing, maintaining and storing the hybridomas to produce monoclonal antibodies in the culture supernatant.

Examples of basal media are low calcium concentration media, such as Ham′F12 medium, MCDB153 medium or low calcium concentration MEM; and high calcium concentration media, such as MCDB104 medium, MEM, D-MEM, RPMI1640 medium, ASF104 medium or RD medium. The basal media can contain, for example, sera, hormones, cytokines, and/or various inorganic or organic substances depending on the objective.

Monoclonal antibodies can be isolated and purified from the culture supernatant or ascites fluid mentioned above by saturated ammonium sulfate precipitation, euglobulin precipitation method, caproic acid method, caprylic acid method, ion exchange chromatography (DEAE or DE52) and affinity chromatography using an anti-immunoglobulin column or a protein A column.

The phrase “a portion of an antibody” in this invention refers to a partial region of a monoclonal antibody such as those described above, and specifically include F(ab′)₂, Fab′, Fab, Fv (variable fragment of an antibody), sFv, dsFv (disulphide stabilized Fv) or dAb (single domain antibody) (Exp. Opin. Ther. Patents, 1996, Vol.6, No.5, p.441-456).

The expression “pharmaceutically acceptable carrier” of this invention includes an excipient, a diluent, a filler, a disintegrating agent, a stabilizer, a preservative, a buffer, an emulsifier, an aromatic agent, a colorant, a sweetener, a thickening agent, a corrigent, a solubility-increasing agent or some other additive.

Using one or more of such carriers, pharmaceutical compositions of the present invention can be formulated into tablets, pills, powders, granules, injections, solutions, capsules, troches, elixirs, suspensions, emulsions, syrups, etc.

The pharmaceutical compositions of the present invention can be administered orally or parenterally. Other forms for parenteral administration include a solution for external application, suppository for rectal administration and pessary, prescribed by the usual method, which comprises one or more active ingredients.

The dosage of the pharmaceutical composition of the present invention can vary depending on the age, sex, weight and symptoms of a patient, effect of treatment, administration route, period of treatment, the kind of active ingredient (the “substance” according to the present invention, mentioned above) contained in the pharmaceutical composition, etc. Usually, the pharmaceutical composition can be administered to an adult at a dose of 10 μg to 1000 mg (or 10 μg to 500 mg) per one administration. Depending on various conditions, a dosage less than that mentioned above may be sufficient in some cases and a dosage more than that mentioned above may be necessary in others.

In the case of an injection, it can be produced by dissolving or suspending an antibody in a non-toxic, pharmaceutically acceptable carrier such as physiological saline or commercially available distilled water for injection adjusting the concentration in the range of 0.1 μg antibody/ml carrier to 10 mg antibody/ml carrier. The injection thus produced can be administered to a human patient in need of treatment in the dose range of 1 μg to 100 mg/kg body weight, preferably in the range of 50 μg to 50 mg/kg body weight, one or more times a day. Examples of administration routes are medically appropriate administration routes such as intravenous injection, subcutaneous injection, intradermal injection, intramuscular injection, intraperitoneal injection or such, preferably intravenous injection.

The injection can also be prepared into a non-aqueous diluent (for example, propylene glycol, polyethylene glycol, vegetable oil such as olive oil, and alcohol such as ethanol), suspension or emulsion.

The injection can be sterilized by filtration with a bacteria-filtering filter, by mixing a bactericide or by irradiation. The injection can be produced in such a manner that it is prepared at the time of use. Namely, it is freeze-dried to be a sterile solid composition that can be dissolved in sterile distilled water for injection or another solvent before use.

The pharmaceutical composition of the present invention can be used alone or in combination with the above-mentioned HGF drug for maintaining or promoting repair, regeneration or neogenesis of tissues and organs required for the prevention and/or treatment of various diseases. Therefore, the use of the pharmaceutical composition alone or in combination with the HGF drug enables treatment of various intractable diseases (for example, hepatic fibrosis, hepatic cirrhosis, fulminant hepatitis, viral hepatitis, acute hepatitis, chronic renal failure, renal fibrosis, pulmonary fibrosis, etc.) which treatment is considered difficult today.

In the combined use of the pharmaceutical composition (drug A) of this invention with the HGF drug (drug B), drug A and drug B can be administered to a patient in any order and any dosage form depending on the purpose of treatment.

When the pharmaceutical composition of this invention is used alone, the biological activity originally possessed by endogenous HGF in the body of a patient is elicited by the active ingredient contained in the pharmaceutical composition, which removes the down-regulation of HGF activity that occurs due to the binding between the HGF receptor c-Met and MERMUC, to accomplish the therapeutic effect.

On the other hand, when the pharmaceutical composition of this invention is used in combination with the HGF drug, the dose of the HGF drug used in treating disorders such as those described above can be kept minimum by the active ingredient contained in the pharmaceutical composition, which removes the down-regulation of HGF activity that occurs due to the binding between HGF receptor c-Met and MERMUC.

In the present invention, “fibrosis” includes tissue fibrosis in any organ, and particularly preferable examples include pulmonary fibrosis, hepatic fibrosis or renal fibrosis.

In the present invention, hepatitis encompasses any inflammation of the liver elicited by any cause, and particularly preferable examples include fulminant hepatitis, acute hepatitis (inflammation elicited by liver toxins and such, due to viruses and pharmaceutical agents) or viral hepatitis (inflammation elicited by various viruses, in particular, hepatitis viruses such as type C hepatitis).

The pharmaceutical composition of the present invention can be used for treating (including preventing) any disorder for which regeneration or neogenesis of tissues is necessary for its treatment, and particularly preferred disorders include various hepatic disorders (e.g., hepatic fibrosis, hepatic cirrhosis, fulminant hepatitis, viral hepatitis, acute hepatitis, etc.), renal disorders (e.g., chronic kidney failure, renal fibrosis, etc.), and pulmonary disorders (e.g., pulmonary fibrosis). Such disorders are all representative intractable diseases, and thus, in other words, the pharmaceutical compositions of this invention are useful for treating or preventing such intractable diseases.

Furthermore, the present invention relates to a “method for identifying a substance having the activity to inhibit the binding between c-Met and MERMUC” (same as the definition indicated above) and a “method for testing the presence or extent of activity of a certain substance to inhibit the binding between c-Met and MERMUC”. Such methods comprise, for example, the following steps of:
(a) preparing a cell that is designed to coexpress c-Met and MERMUC, and upon binding of c-Met and MERMUC, allow detection of their binding;
(b) culturing the cell obtained in step (a) in the presence of HGF while in the absence of test substance, and then measuring the extent of the binding between c-Met and MERMUC in the cell;
(c) culturing the cell obtained in step (b) in the presence of HGF and a test substance, and then measuring the extent of the binding between c-Met and MERMUC in the cell; and
(d) comparing the measurement result of step (b) with that of step (c).

Herein, host cells used for producing the cell of step (a) are not particularly limited as long as they are cells normally used for the production of recombinant proteins, and include any of the various cells of such as natural cells or artificially established recombinant cells (for example, bacteria (Escherichia, Bacillus, etc.), yeast (Saccharomyces, Pichia, etc.), animal cells, insect cells, etc.). More specifically, it may be any one of E. coli (DH5α, TB1, HB101, etc.), mouse-derived cells (COP, L, C127, Sp2/0, NS-1, NIH3T3, etc.), rat-derived cells (PC12, PC12h), hamster-derived cells (BHK, CHO, etc.), monkey-derived cells (COS1, COS3, COS7, CV1, Velo, etc.) and human-derived cells (HeLa, diploid fibroblast-derived cells, myeloma cells, HepG2, etc.). Animal cells such as those mentioned above are particularly preferred.

A favorable example of c-Met (full-length or a portion thereof) of step (a) includes a fusion polypeptide formed between all or a part of human c-Met defined above (a particularly preferred example includes those containing at least the binding site with human MERMUC) and β-galΔα or β-galΔω.

A favorable example of MERMUC (full-length or a portion thereof) of step (a) includes a fusion polypeptide formed between all or a part of human MERMUC defined above (a particularly preferred example includes those containing at least the binding site with human c-Met) and Δ-galΔα or Δ-galΔω.

By using the fusion polypeptide mentioned above, the presence, extent, or increase or decrease of the binding with MERMUC can be detected as β-galactosidase activity expressed within the cell sample (Proc. Natl. Acad. Sci. USA., 1997, Vol.94, p.8405-8410).

Identification or testing of compounds using the method of this invention can be performed manually. However, a quicker and more easy identification or testing can be achieved by using the so called high throughput screening, which is performed automatically using a machine (robot) (Saibo Baiyo Kogaku (Tissue Culture Engineering), 1997, Vol.23, No.13, p.521-524; U.S. Pat. No. 5,670,113).

Substances identified by the method of the present invention are used as active ingredients of the pharmaceutical composition of this invention.

### BRIEF DESCRIPTION OF THE DRAWINGS

FIG. 1 depicts photographs showing the production of human MERMUC and/or human c-Met in recombinant human MERMUC-expressing HEK293 cells analyzed by Western blotting.
(A) depicts a photograph showing the production of human MERMUC in recombinant cells cultured in the presence or absence of Dox analyzed by Western blotting using anti-MERMUC antibody.
(B) depicts a photograph showing the production of human c-Met and MERMUC (coprecipitated with c-Met) in recombinant cells cultured in the presence or absence of Dox analyzed by Western blotting using anti-c-Met or anti-MERMUC antibody.
(C) depicts a photograph showing the production of human c-Met and MERMUC (coprecipitated with c-Met) in recombinant cells cultured in the presence or absence of Dox analyzed by Western blotting using a control antibody.

FIG. 2 depicts photographs showing the production of multimerized human MERMUC in recombinant human MERMUC-expressing HEK293 cells analyzed by Western blotting.
(A) depicts a photograph showing the production of FLAG-tagged human MERMUC in various samples analyzed by Western blotting using anti-FLAG-tag antibody.
(B) depicts a photograph showing the production of poly HIS-tagged human MERMUC in various samples analyzed by Western blotting using anti-poly HIS-tag antibody.

FIG. 3 depicts photographs showing the expression of human MERMUC mRNA in various human tissues analyzed by Northern blotting.

FIG. 4 depicts a photograph showing the expression of human MERMUC gene in various human tissues analyzed by PCR.

FIG. 5 depicts photographs showing the distribution of mouse MERMUC gene expression in various types of mouse kidney tissues analyzed by in situ hybridization. The letters G, PT and DT in the figure indicate glomerulus, proximal tubule and distal tubule, respectively.
(a) indicates the expression distribution of MERMUC gene in kidney tissue of a MRL-lpr/lpr mouse (5 weeks old) analyzed using antisense cRNA probe.
(b) indicates the expression distribution of MERMUC gene in kidney tissue of a MRL-lpr/lpr mouse (5 weeks old) analyzed using sense cRNA probe.
(c) indicates the expression distribution of MERMUC gene in kidney tissue of a MRL-lpr/lpr mouse (23 weeks old) analyzed using antisense cRNA probe.
(d) indicates the expression distribution of MERMUC gene in kidney tissue of a C57BL/6 mouse (23 weeks old) analyzed using antisense cRNA probe.
(e) indicates the expression distribution of MERMUC gene in kidney tissue of a C57BL/6 mouse (23 weeks old) analyzed using sense cRNA probe.

FIG. 6 shows the results of evaluating the binding of MERMUC with HGF receptor c-Met using the expression of β-gal activity as an indicator.

FIG. 7 shows the results of evaluating the intermolecular binding of MERMUC using the expression of β-gal activity as an indicator.

FIG. 8 depicts photographs showing the decrease in HGF-induced intracellular MAP kinase activity due to the increase in the production of MERMUC analyzed by Western blotting.

FIG. 9 depicts a photograph showing the difference in phosphorylated HGF receptor c-Met production between HGF-stimulated MERMUC producing cells and non-producing cells analyzed by Western blotting.

FIG. 10 depicts a photograph showing the difference in HGF receptor c-Met-bound Grb2 and Gab1 production between HGF-stimulated MERMUC producing cells and non-producing cells analyzed by Western blotting.

FIG. 11 depicts photographs showing the difference in MMP-1 and MMP-9 mRNA expressions between HGF stimulated MERMUC producing cells and non-producing cells analyzed by RT-PCR.

FIG. 12 shows the degree of cell proliferation in response to HGF or EGF in hepatocytes derived from MERMUC overexpressing transgenic mice and normal mice, respectively.
(A) indicates the degree of cell proliferation in response to HGF.
(B) indicates the degree of cell proliferation in response to EGF.

FIG. 13 depicts photographs showing the intensity of HGF-induced MAP kinase activity in hepatocytes of MERMUC overexpressing transgenic mice and normal mice, respectively, analyzed by Western blotting.

FIG. 14 shows the results of evaluating the presence of self-association of mFas-MERMUC fusion protein at MERMUC region using the expression of β-gal activity as an indicator.

FIG. 15 depicts photographs showing the amount of self-associated MERMUC co-precipitating with the HGF receptor analyzed by Western blotting.

FIG. 16 shows the results of evaluating the degree of suppression of MERMUC self-association due to various MERMUC cytoplasmic region peptides using the expression of β-gal activity as an indicator.

FIG. 17 shows the results of evaluating the degree of suppression of MERMUC binding to HGF receptor c-Met due to various MERMUC cytoplasmic region peptides using the expression of β-gal activity as an indicator.

FIG. 18 shows the results of evaluating the degree of suppression of self-association of MERMUC and the binding of MERMUC to HGF receptor c-Met due to various concentrations of MERMUC cytoplasmic region peptide (del-4) using the expression of β-gal activity as an indicator.

FIG. 19 depicts photographs showing the existence of MERMUC expression in the presence or absence of Dox, and the intensity of phosphorylated MAP kinase (p-Erk) production depending on the presence or absence of suppression of MERMUC self-association due to the MERMUC cytoplasmic region peptide (del-4) analyzed by Western blotting.

FIG. 20 depicts photographs showing the existence of MERMUC expression in the presence or absence of Dox and/or the existence of phosphorylated MERMUC production in the presence or absence of HGF stimulation.

FIG. 21 depicts photographs showing the difference in phosphorylated MERMUC productions in the presence or absence of HGF and/or HGF signaling suppressor by immunoprecipitation.

FIG. 22 shows the degree of HGF-dependent liver enlargement in MERMUC over expressing Tg and normal mice.

### BEST MODE FOR CARRYING OUT THE INVENTION

The present invention will be described in detail below with reference to the Examples, but is not to be construed as being limited thereto.

### EXAMPLE 1

### Identification of Molecules Binding to Human MERMUC

Human genome analysis revealed that the gene encoding human MERMUC exists on chromosome 3 and comprises 3 exons (Ex.-1, Ex.-2 and Ex.-3).

The primary structure (amino acid sequence) of every exon in human and mouse MERMUC polypeptides were compared. The homology between human and mouse at the region corresponding to Ex.-1 was low, of about 30%, however, that at the regions corresponding to Ex.-2 and Ex.-3 (each region comprises approximately 50 amino acids) were extremely high, i.e., 71% and 60%, respectively. Therefore, based on the analysis of the primary structure of the MERMUC peptides alone, these regions Ex.-2 and Ex.-3 corresponding to the C-terminal region of the MERMUC cytoplasmic region were suggested to be involved in some kind of important role for the expression of MERMUC function.

Thus, to reveal the function of MERMUC, the clarification of existence and identification of molecules that bind to the C-terminal region of MERMUC cytoplasmic region was performed.

Specifically, yeast two hybrid system (Proc. Natl. Acad. Sci. USA., 1991, Vol.88, p.9578-9582) was performed to screen a human kidney cDNA library using cDNA corresponding to the C-terminal region of the human MERMUC as the bait (that is, fishing bait).

Two kinds of bait (bait-2 and bait-3) were designed using each of Ex.-2 and Ex.-3 as the core. Bait-2 corresponds to a 123 amino acid portion of the C-terminus (bait-2) and bait-3 corresponds to the 65 C-terminal amino acids (bait-3).

Both baits were prepared by polymerase chain reaction (PCR) according to conventional methods using the full-length cDNA encoding human MERMUC as the template. Both of the two forward primers (<bait-2> SEQ ID NO: 18 and <bait-3> SEQ ID NO: 19) used for the PCR contain an EcoRI site at their 5′ ends and a reverse primer (SEQ ID NO: 20) common to both baits contained a SalI site at its 5′ end.

Each bait vector was obtained by inserting each DNA fragment obtained by digesting each of the PCR products with the restriction enzymes EcoRI and SalI into the EcoRI/SalI site of pGBKT7 vector (leucine biosynthesis gene as a marker; CLONTECH). In yeast, the gene within the vector is produced as a fusion protein ([fusion protein A]) with the DNA binding region (GAL4-DB) of the yeast transcription factor GAL4.

On the other hand, each cDNA of the human kidney derived cDNA library (CLONTECH) that is used for the screening with the bait vector is inserted in pADK vector (tryptophan biosynthesis gene as a marker). Therefore, proteins produced through the expression of each of these cDNAs in yeast are produced as a fusion protein ([fusion protein B]) with the GAL4 transcriptional activation region (GAL4-AD).

AH109 strain (leucine, tryptophan, histidine and adenine auxotrophic yeast; CLONTECH) was used as the yeast host. This yeast strain contains 3 types of genes (histidine biosynthesis gene, adenine biosynthesis gene and β-galactosidase gene) under the control of the GAL promoter on the chromosome.

The function as a transcription factor is recovered through the fusion of GAL4-DB and GAL4-AD by the binding of the bait protein (the fusion protein A) with the prey protein (the fusion protein B). As a result, the expression of the 3 genes is induced to supplement the histidine and adenine requirement, and exhibit β-galactosidase (β-gal) activity.

Each bait vector was introduced into yeast AH109 strain with the cDNA library plasmid mentioned above (50,000 clones per pool) by the polyethylene glycol/lithium acetate method for screening (J. Bacteriol., 1983, Vol. 153, p.163-16883; Curr. Genet., 1989, Vol. 16, p. 339-346; Nucleic Acids Res., 1991, Vol. 19, p. 5791; Nucleic Acids Res., 1992, Vol. 20, p.1425).

The marker for the first selection of yeast with the desired transformation in the present screening was acquired histidine non-requirement due to the expression of the histidine biosynthesis gene. Therefore, after introducing the plasmid, yeast was cultured for 5 days at 30° C. in yeast minimum medium that lacks leucine, tryptophan and histidine (SD/-Leu,-Trp,-His) used for selecting transformants.

Transformed yeast that grew on the plate upon cultivation were picked with toothpick and seeded on 3 kinds of plates described below, and then cultured for 5 days at 30° C. for a second selection of the desired clones:
<A> plate containing yeast minimum medium lacking leucine, tryptophan and histidine (SD/-Leu, -Trp, -His);
<B> plate containing yeast minimum medium lacking leucine, tryptophan and adenine (SD/-Leu, -Trp, -Ade); and
<C> plate containing yeast minimum medium lacking leucine and tryptophan, and supplemented with α-X-gal (20 ug/ml) (SD/-Leu, -Trp, +X-gal).

Positive clones were identified as colonies growing on both of the above-mentioned <A> and <B> plates (acquirement of histidine and adenine non-requirement) and showing blue color on plate <C> (α-X-gal addition) (expression of β-galactosidase activity). Four prey cDNA clones (clones No. 3-309, No. 3-312, No. 2-22 and No. 2-211) were selected as positive. Plasmids possessing prey cDNA fragments carried by the positive yeast clones were transferred into E. coli from the yeast by the method described by Marcil and Higgins (Nucleic Acids Res., 1992, Vol. 20, p. 917).

The nucleotide sequences of the DNA fragments corresponding to the prey cDNAs were determined through nucleotide sequence determination procedure by the dideoxy method after recovering the plasmid DNAs from E. coli. Based on the determined nucleotide sequences of the prey cDNAs, searching and data analysis were performed using widely used gene databases.

As a result, the proteins derived from two clones (No. 3-309 and 3-312) among the four positive clones had binding ability to both the proteins derived from bait-2 and bait-3. Moreover, the proteins corresponded to the C-terminal region of the β chain of c-Met that is the human HGF receptor. Clones No. 3-309 and No. 3-312 corresponded to the C-terminal 91 amino acids (amino acids 993-1083 of SEQ ID NO: 4) and the C-terminal 89 amino acids (amino acids 995-1083 of SEQ ID NO: 4) of the human c-Met β chain, respectively.

Moreover, proteins derived from two other positive clones (No. 2-22 and 2-211) had binding ability to the protein derived from bait-2 and corresponded to the C-terminal region of human MERMUC itself. Clones No. 2-22 and No. 2-211 corresponded to the C-terminal 93 amino acids (amino acids 411-503 of SEQ ID NO: 10) and the C-terminal 56 amino acids (amino acids 448-503 of SEQ ID NO: 10) of human MERMUC, respectively.

### EXAMPLE 2

### Binding of Full-Length MERMUC Protein and Full-Length HGF Receptor c-Met Protein

<2-1> Preparation of MERMUC Expressing Recombinant Cells Whose Expression can be Regulated by a Drug

A recombinant cell that can regulate the expression of human MERMUC gene by Doxicycline (Dox) was prepared using the reported Tetracycline-controllable expression system (Tet system). Specifically, the recombinant cell possess a characteristic (Tet-off system) wherein the expression of the MERMUC gene is regulated depending on the concentration of Dox contained in the medium.

The cell was prepared using a commercially available pBI-L vector, pTK-Hyg vector and HEK293 Tet-Off cell (all from CLONTECH) and experimental procedures followed the experimental protocol provided with the experimental tool.

Specifically, a MERMUC expression vector was prepared by inserting cDNA (SEQ ID NO: 9) encoding the full-length human MERMUC into the multicloning site of the pBI-L vector. Next, HEK293 Tet-Off cell was cotransfected with the MERMUC expression vector and the pTK-Hyg vector (HEK293 cell strain derived from human kidney expresses the human HGF receptor c-Met). Cell strains stably transfected with the MERMUC expression vector were selected using the presence of hygromycin resistance as an indicator. Then, a cell strain (dubbed MERMUC/203 cell) with high MERMUC gene expression efficiency and having a characteristic to enable precise regulation of the expression by DOX were further selected from the selected cell group. The selection was performed through the analysis of the expressed MERMUC protein by Western blotting using anti-human MERMUC polyclonal antibody.

<2-2> Analysis of Binding Between Human MERMUC and Human HGF Receptor c-Met by Immunoprecipitation

Immunoprecipitation was performed by adding anti-c-Met antibody into the cell lysate prepared from MERMUC/293 cells as described above. The binding between full-length MERMUC protein with full-length HGF receptor c-Met protein was evaluated whether they coimmunoprecipitate or not.

Polyclonal antibody against human MERMUC used in the present assay was prepared according to a conventional method by immunizing a rabbit with a peptide corresponding to the 17 amino acids (SEQ ID NO.21) near the N-terminus. On the other hand, commercially available polyclonal antibody h-Met (C-28) (sc-161; Santa Cruz Biotechnology) was used as human c-Met antibody.

The anti-human c-Met polyclonal antibody was added to the cell lysate prepared from MERMUC/293 cells for reaction. The reaction solution was reacted with microbeads-bound protein-G Sepharose, and then centrifuged to immunoprecipitate the complex of c-Met and anti-c-Met polyclonal antibody.

The precipitate was then dissolved in electrophoresis buffer and the sample dissolved in the buffer was separated by SDS polyacrylamide gel electrophoresis (SDS-PAGE). Then, Western blotting was performed using anti-human MERMUC polyclonal antibody according to conventional method.

As a result, the existence of human MERMUC was detected (FIG. 1) in the proteins immunoprecipitated with anti-human c-Met polyclonal antibody. This result reproduces the binding of full-length MERMUC with full-length HGF receptor c-Met in actual cells, and indicates the possibility that MERMUC modulates the signal transduction via the HGF/HGF receptor through the binding to c-Met.

### EXAMPLE 3

### Analysis of Intermolecular Bindings (Multimerization) of Human MERMUC by Immunocoprecipitation

An intermolecular binding activity between MERMUC molecules was predicted from the result of Example 1. Therefore, the existence of intermolecular binding of full-length MERMUC protein was analyzed.

<3-1> Construction of Full-Length Human MERMUC Expression Vector

Expression vectors expressing full-length human MERMUC with two different tags were constructed as follows. One vector expresses MERMUC added with a FLAG-tag at the C-terminus of MERMUC and the other expresses poly HIS-tagged MERMUC.

cDNAs encoding each of the tagged full-length MERMUC proteins were prepared by conventional PCR method using cDNA (SEQ ID NO: 9) encoding the full-length human MERMUC. Primer sets used for the PCR were as follows:

<FLAG-tagged MERMUC>
forward primer: SEQ ID NO: 22, and
reverse primer: SEQ ID NO: 23; and

<polyHIS-tagged MERMUC>
forward primer: SEQ ID NO: 22, and
reverse primer: SEQ ID NO: 24.

Each of the obtained PCR products was digested with restriction enzymes HindIII and XhoI, inserted into the HindIII and XhoI site of pcDNA3 vector, and then transformed into E. coli. Each plasmid for mammalian cell expression used in the present assay was prepared from the transformed E. coli.

A mixture containing equal amounts of each of the two expression vectors prepared above was added and cultured at 37° C. for 48 hr to cotransfect HEK293 cells (human kidney derived cell strain) using GeneJammer Transfection Reagent (STRATEGENE) following the experimental protocol provided with the reagent.

The cell lysate prepared from the obtained transfected cells was treated for immunoprecipitation with anti-polyHIS polyclonal antibody (Santa Cruz Biotechnology), anti-FLAG monoclonal antibody (Anti-FLAG M2 monoclonal antibody; #F3165; Sigma) or control monoclonal antibody.

Next, the presence of each of the FLAG-tagged MERMUC and polyHIS-tagged MERMUC in the immunoprecipitated protein was analyzed by Western blotting using anti-FLAG monoclonal antibody and anti-polyHIS polyclonal antibody according to conventional method.

The results are shown in FIG. 2.

As a result, both of the FLAG-tagged MERMUC and polyHIS-tagged MERMUC were identified when anti-FLAG monoclonal antibody was used for immunoprecipitation. Furthermore, both of the tagged MERMUC were also identified when anti-poly HIS polyclonal antibody was used for immunoprecipitation.

These results indicate that MERMUC forms a multimer (multimerizes) via intermolecular binding of monomers on the cell membrane.

### EXAMPLE 4

### Tissue Distribution of Human MERMUC Expression

The expression of human HGF receptor c-Met is reported to be particularly high not only in liver, kidney and prostate, but also in digestive organs, such as small intestine and large intestine (Int. J. Cancer, 1991, Vol. 49, p. 323-328; Oncogene, 1991, Vol. 6, p. 1977-2003).

<4-1> Analysis by Northern Blot Method

Thus, tissue distribution of MERMUC expression was analyzed by Northern blotting according to conventional method in order to analyze the physiological importance of the binding between MERMUC and HGF receptor c-Met.

Three commercially available membranes (Human 12-lane MTN Blot, Human MTN Blot II and Human Digestive MTN Blot; all from CLONTECH) blotted with human tissue derived mRNAs (2 μg/lane) were used. Furthermore, a DNA fragment (about 1.5 kbp) comprising the cDNA region encoding the full-length human MERMUC that was prepared through the amplification by PCR was used as a probe. Hybridization was performed at 65° C. over night. Membranes were washed with 4×SSC (0.1% SDS) for 15 min, and 2×SSC (0.1% SDS) for 15 min after the hybridization.

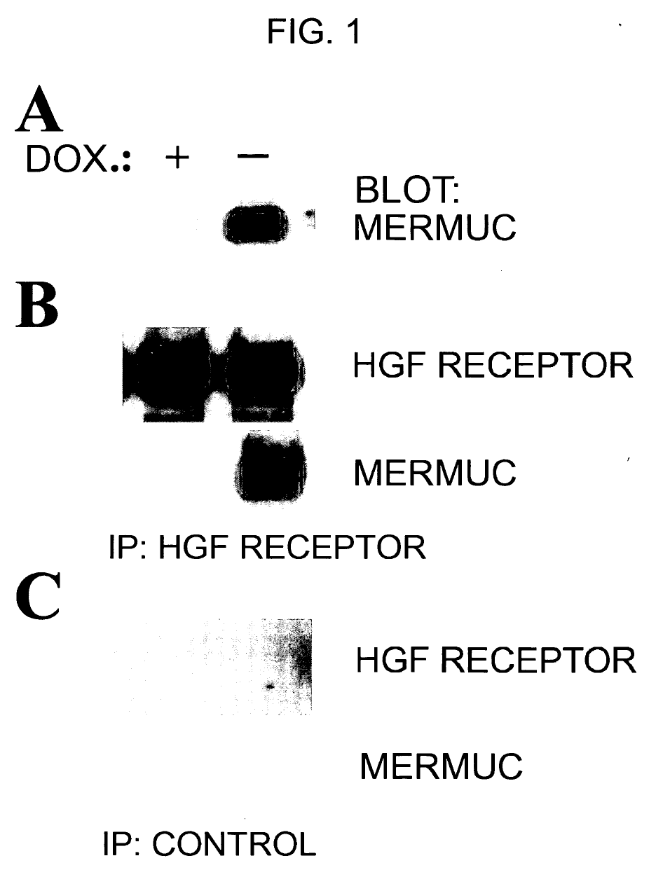

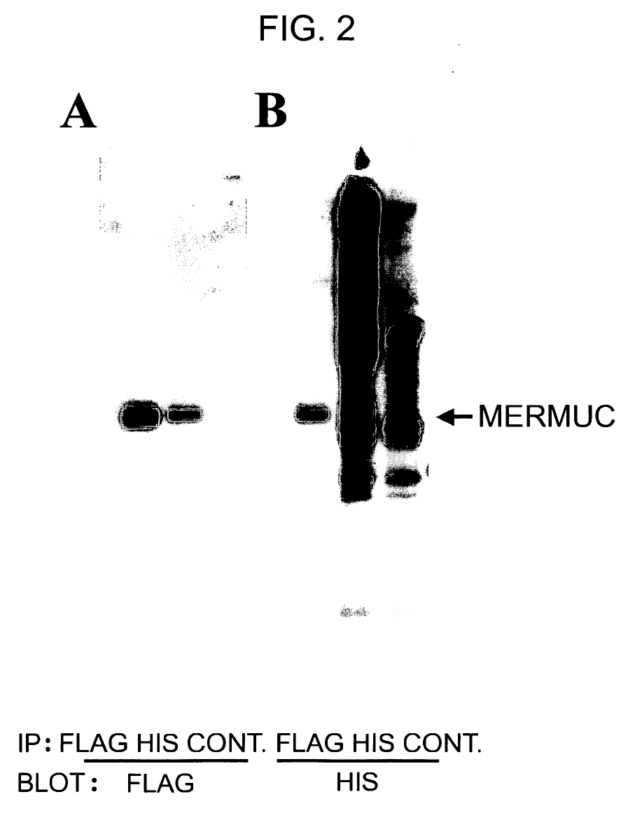

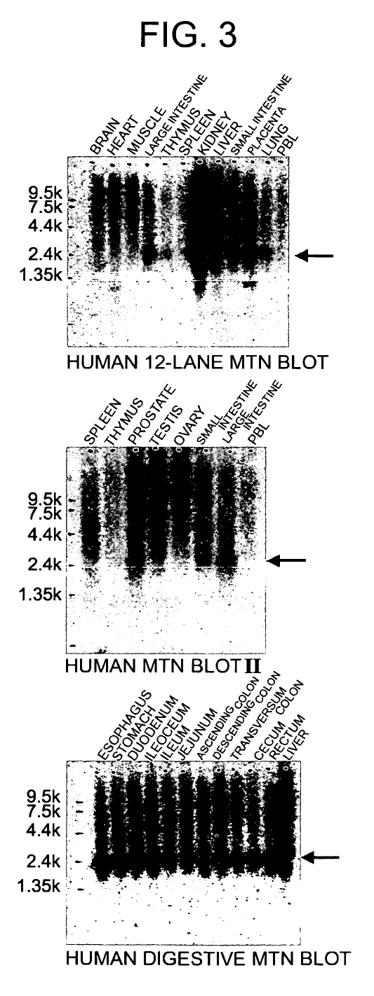

The results are shown in FIG. 3

A band of approximately 2.4 kbp, corresponding to MERMUC mRNA was detected in kidney, prostate, liver, lung, placenta, and digestive organs such as small intestine and large intestine. This expression distribution matched with that of the HGF receptor c-Met.

<4-2> Analysis by PCR Method

Furthermore, the expression of the MERMUC gene in some of the organs was analyzed by methods utilizing PCR.

Specifically, analyses were conducted by PCR (35 cycles at the annealing temperature 55° C.) using cDNAs as a template (0.2 ng each; Human Multiple Tissue cDNA Panel I; CLONTECH) that were derived from human brain, heart, liver, kidney, lung, pancreas, placenta and skeletal muscle, respectively and forward primer (SEQ ID NO: 25; corresponding to a part of Ex.-2) and reverse primer (SEQ ID NO: 26; corresponding to a part of Ex.-3).

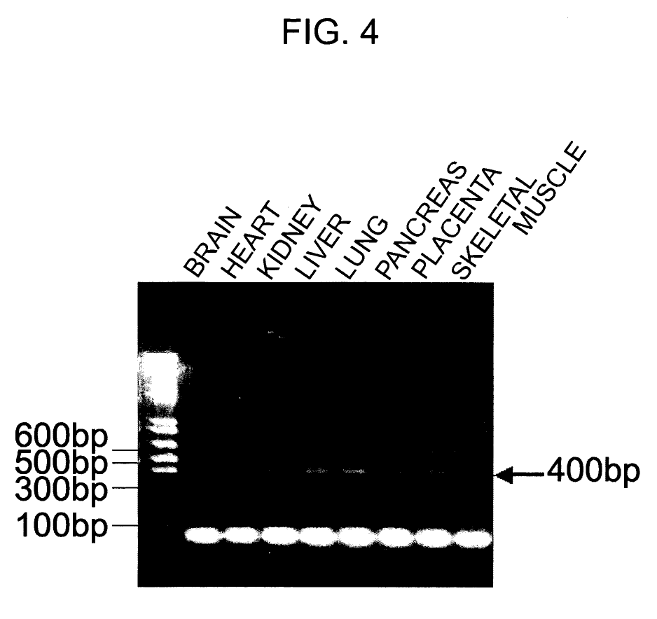

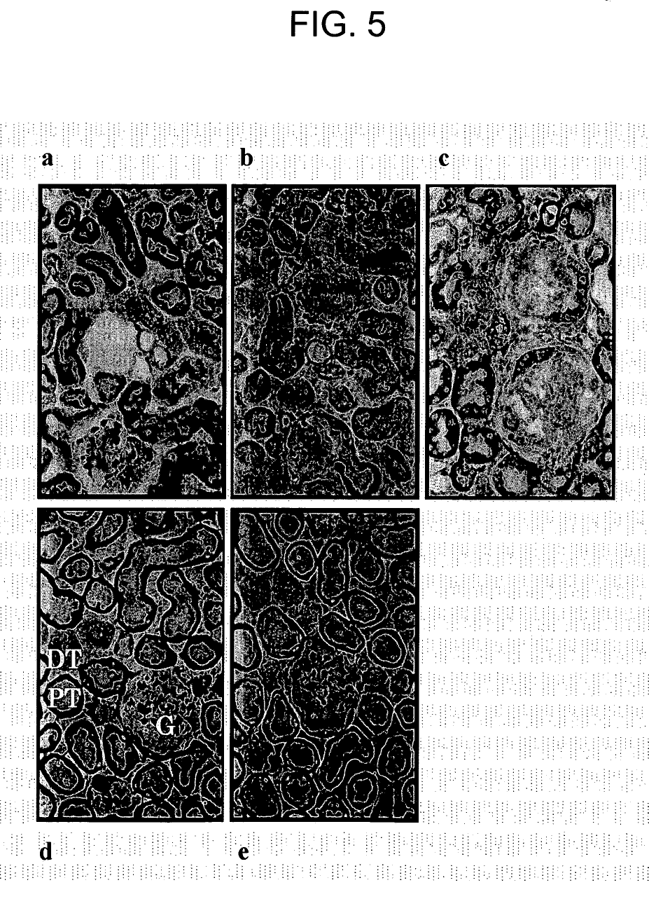

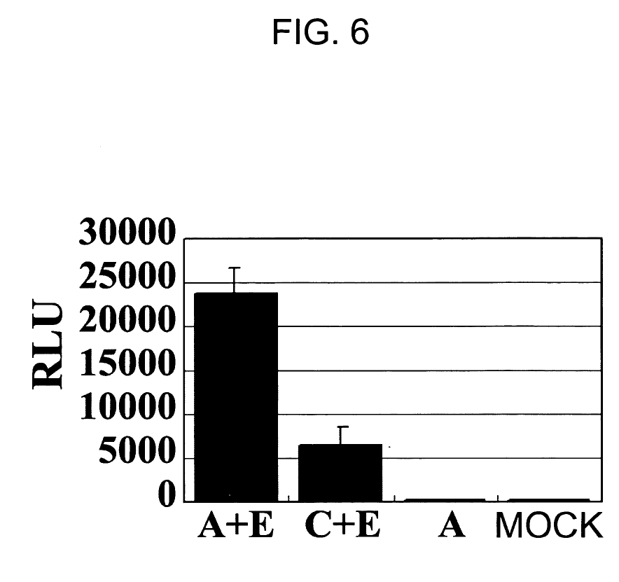

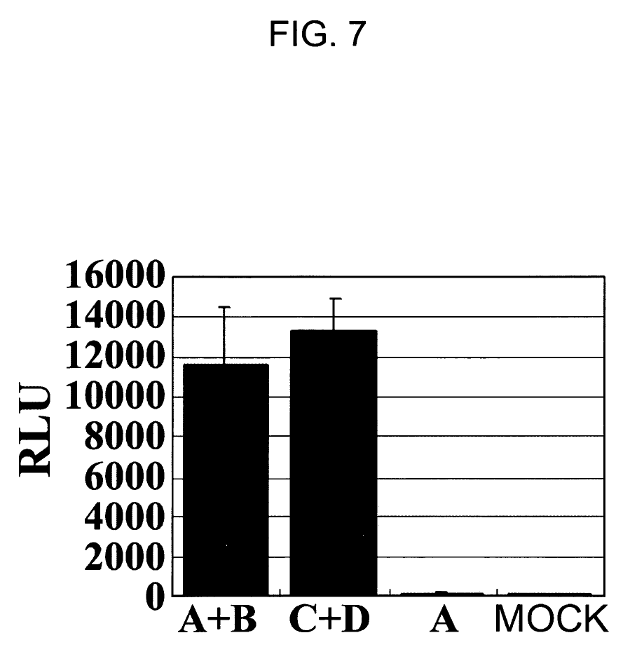

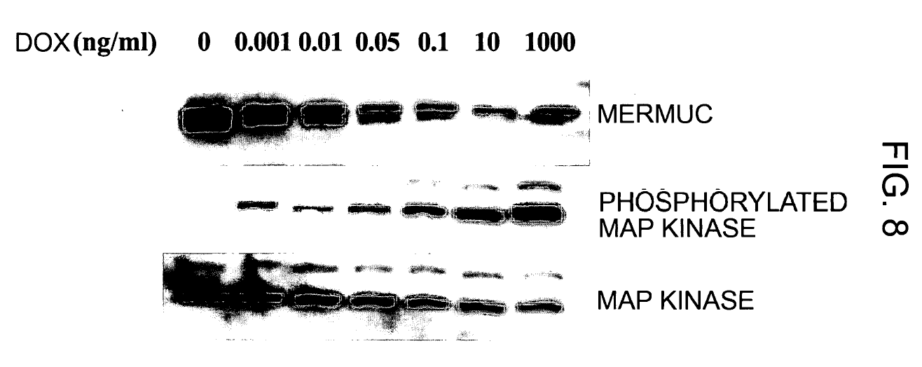

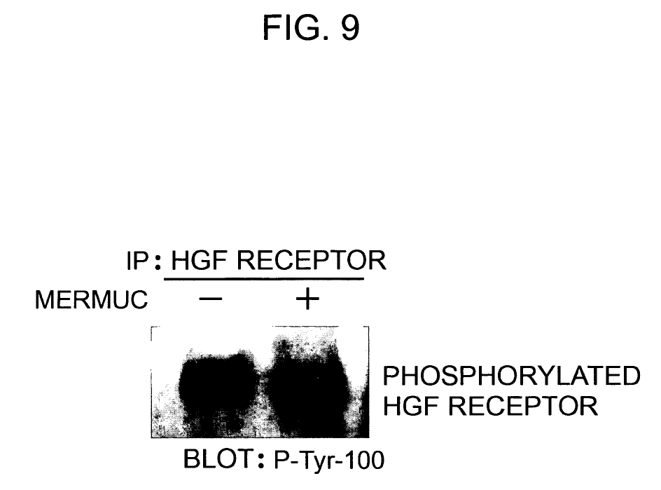

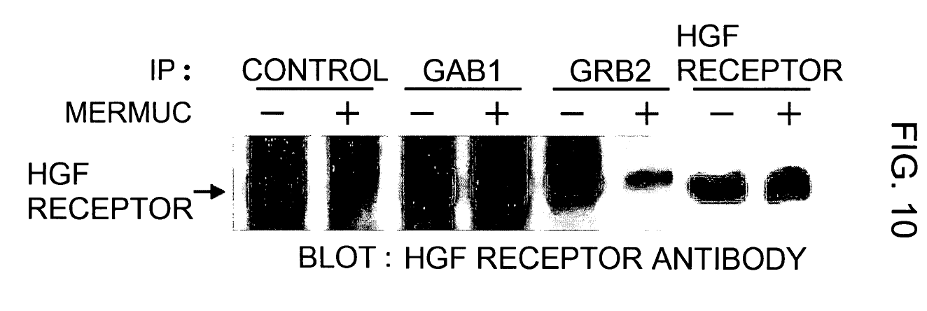

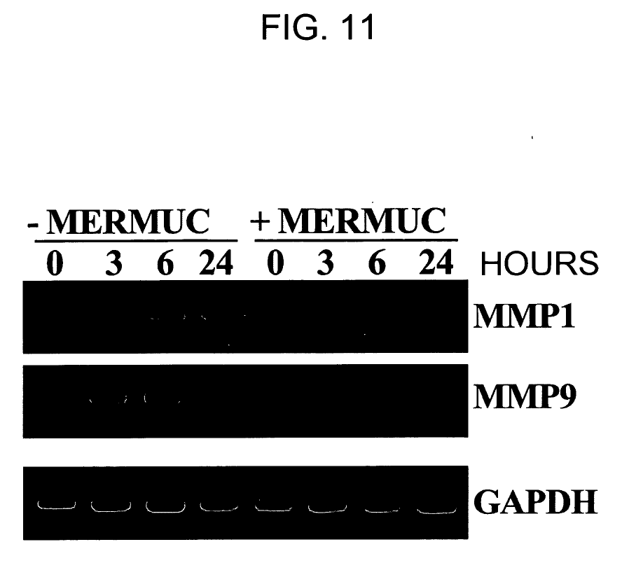

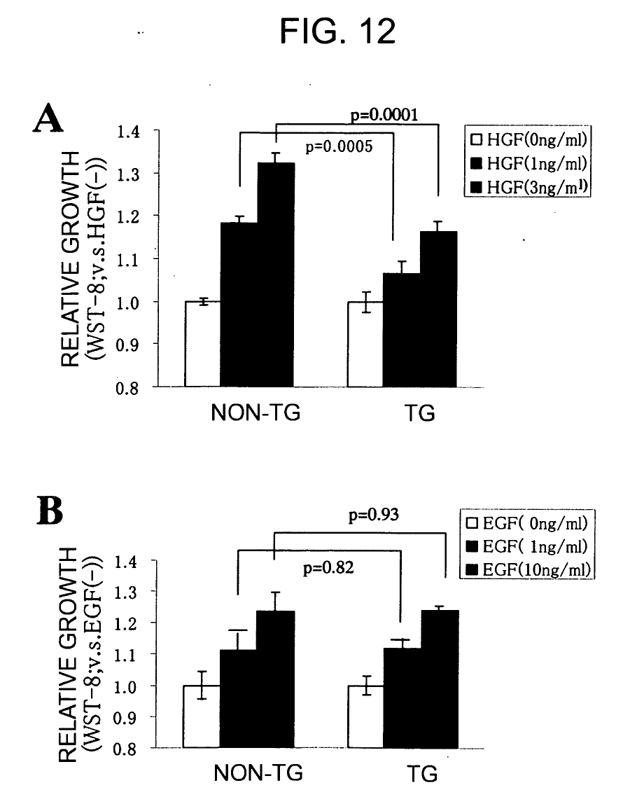

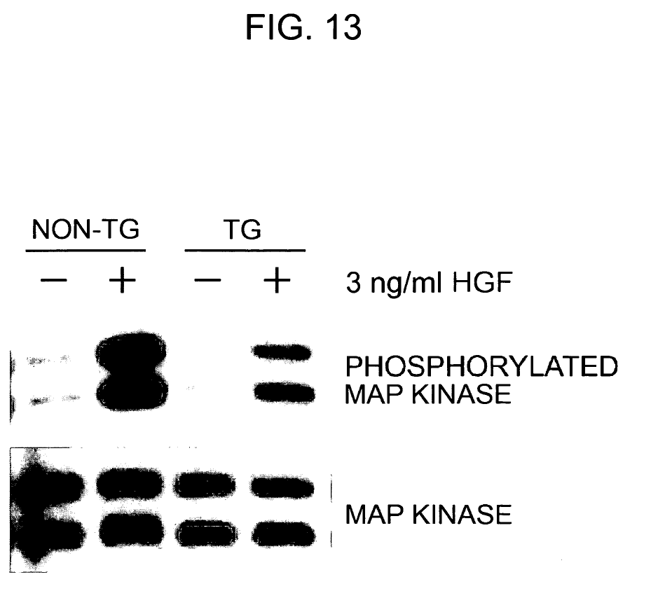

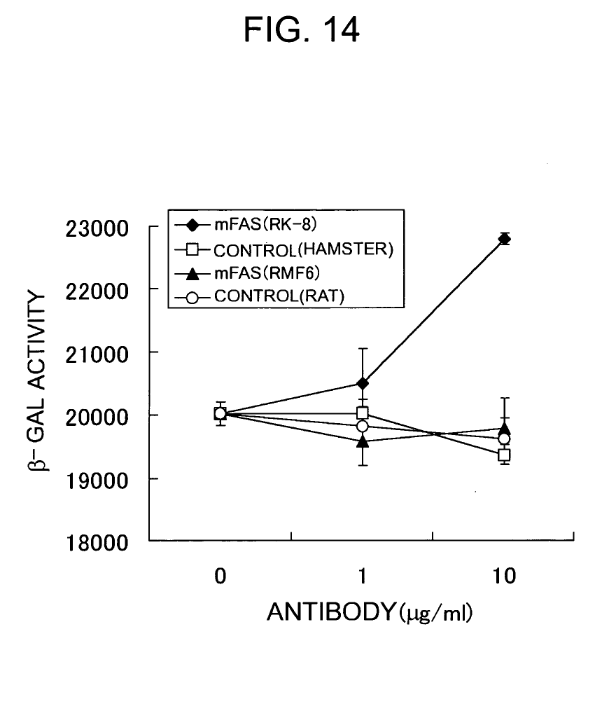

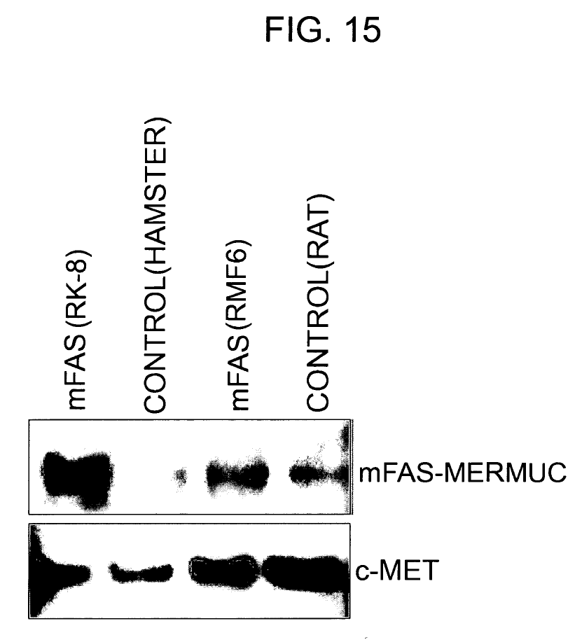

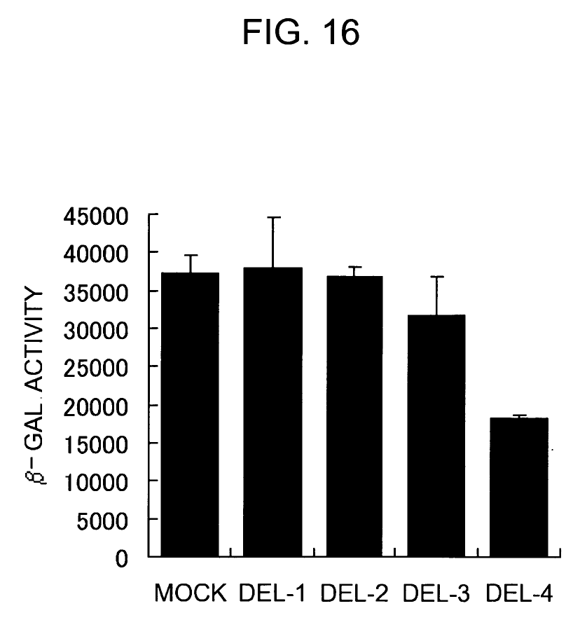

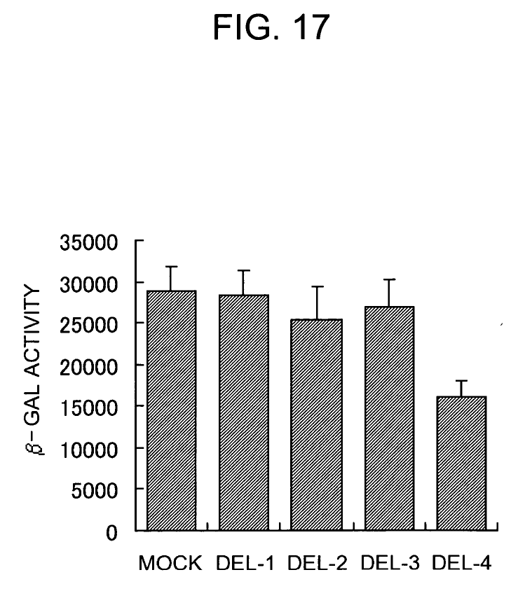

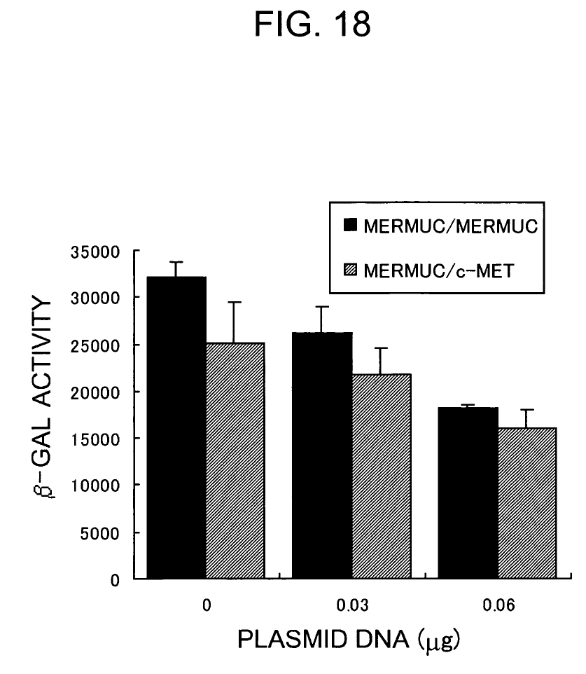

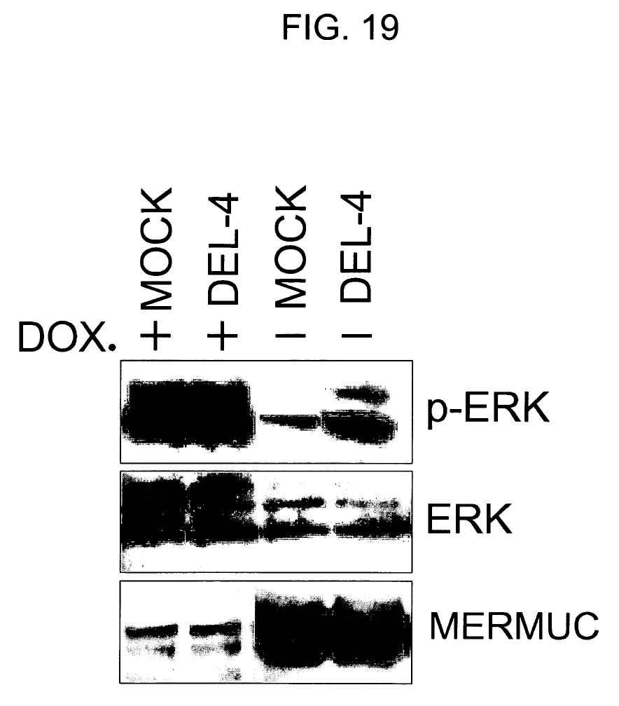

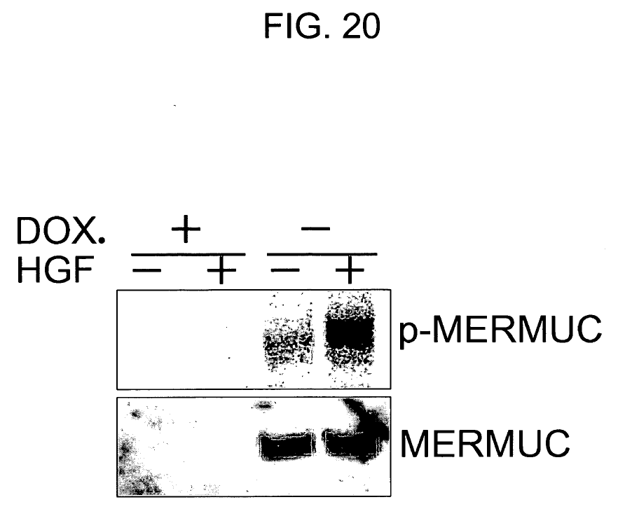

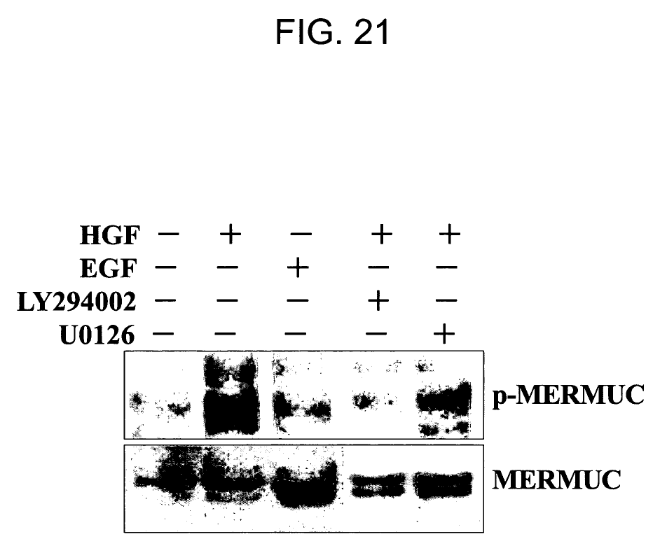

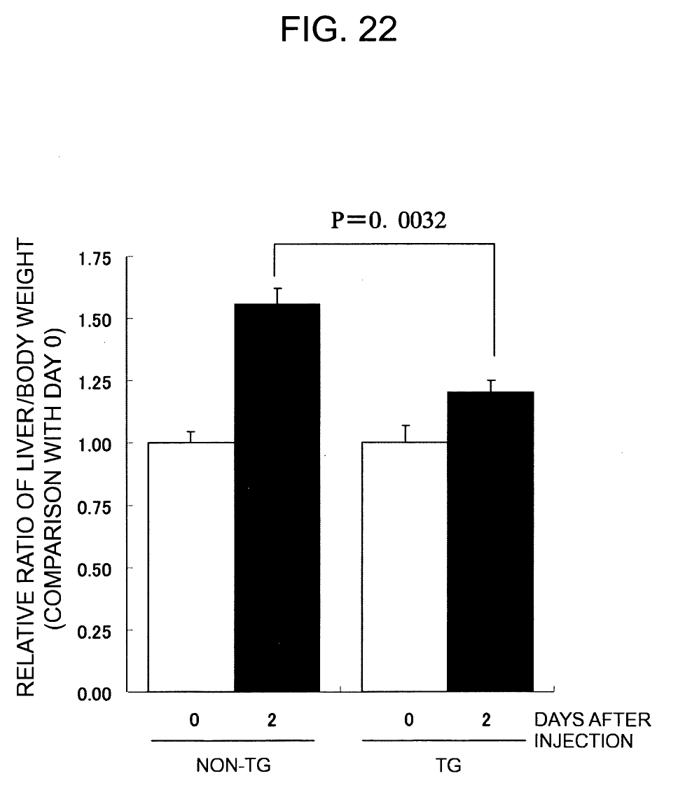

The results are shown in FIG. 4. As a result, an amplified DNA band of approximately 400 bp that indicate the expression of MERMUC gene was detected in kidney, liver, lung, placenta and pancreas confirming significant expression of MERMUC gene in these organs.

<4-3>Analysis by in situ Hybridization

The expression of the HGF receptor c-Met gene in kidney tissue is localized in the epithelial cells of renal urinary tubule, and is involved in cell proliferation and lumen formation (Biochem. Biophys. Res. Commun., 1991, Vol. 174, p831-838; Cell, 1991, Vol. 67, p.901-908). Thus, the distribution of mouse MERMUC gene expression in kidney tissues was analyzed by in situ hybridization.

Tissue sections for RNA hybridization were prepared from kidney tissues of commercially available MRL-lpr/lpr mice (5 or 23 weeks old) and C57BL/6 mice (23 weeks old) according to a conventional method.

Anti-sense cRNA probe or sense cRNA probe synthesized using a fragment of about 500 bp encoding the N-terminus of the full-length mouse MERMUC (amino acid/SEQ ID NO: 11; cDNA/SEQ ID NO: 12) as a template was used for the in situ hybridization.

The results are shown in FIG. 5.

As a result, the expression of mouse MERMUC gene in kidney tissue was localized in the epithelial cells of the distal proximal tubules. The result agreed with the reported distribution of the HGF receptor c-Met expression in kidney cells.

### EXAMPLE 5

### Identification of Human HGF Receptor c-Met Intramolecular Binding Region to Human MERMUC Molecule

The present invention was performed using the reported yeast two-hybrid system (Proc. Natl. Acad. Sci. USA., 1991, Vol. 88, p.9578-9582) based on conventional method and the experimental protocols appended to the reagents.

PCR was performed using DNA (clone No. 3-309) corresponding to the 91 amino acids (amino acids 993-1083 of SEQ ID NO: 4) of the C-terminal region of the human HGF receptor c-Met β chain that was identified in the previous Example to have the binding ability to the cytoplasmic region of human MERMUC as a template. Seven DNA fragments shown below corresponding to the C-terminal region of the c-Met β chain having various lengths, wherein various length of N-terminal or C-terminal amino acids are deleted from said 91 amino acids were prepared via a conventional method by PCR:
A. DNA corresponding to a region (amino acids 1003-1083 of SEQ ID NO: 4) that lacks the 10 N-terminal amino acids of the 91 amino acids of the C-terminal human c-Met β chain;
B. DNA corresponding to a region (amino acids 1013-1083 of SEQ ID NO: 4) that lacks the 20 N-terminal amino acids of the 91 amino acids of the C-terminal human c-Met β chain;
C. DNA corresponding to a region (amino acids 1023-1083 of SEQ ID NO: 4) that lacks the 30 N-terminal amino acids of the 91 amino acids of the C-terminal human c-Met β chain;
D. DNA corresponding to a region (amino acids 993-1073 of SEQ ID NO: 4) that lacks the C-terminal 10 amino acids of the 91 amino acids of the C-terminal human c-Met β chain;
E. DNA corresponding to a region (amino acids 993-1063 of SEQ ID NO: 4) that lacks the 20 C-terminal amino acids of the 91 amino acids of the C-terminal human c-Met β chain;
F. DNA corresponding to a region (amino acids 993-1053 of SEQ ID NO: 4) that lacks the 30 C-terminal amino acids of the 91 amino acids of the C-terminal human c-Met β chain; and
G. DNA corresponding to a region (amino acids 993-1043 of SEQ ID NO: 4) that lacks the 40 C-terminal amino acids of the 91 amino acids of the C-terminal human c-Met β chain.

Furthermore, the forward and reverse primers used for the PCR were designed to contain an EcoRI site and SalI site at their 5′ end, respectively. Each of the DNA fragments prepared by PCR were inserted into the EcoRI/SalI site of pADKT7 vector (a vector for a prey protein that contains a tryptophan biosynthesis gene as a marker; CLONTECH) in frame with GAL4-AD to prepare prey vectors wherein each of the 7 DNAs described above is inserted.

Each of the 7 prey vectors was cointroduced into yeast AH109 strain with the human MERMUC bait-2 vector prepared in Example 1.

The binding activity between the c-Met prey proteins derived from the 7 DNA fragments and the MERMUC bait protein was analyzed by the yeast two-hybrid system described in Example 1. Specifically, the binding activity was evaluated based on the growth of yeast transformed with the vector on 2 kinds of yeast minimum media, i.e., (1) SD/-Leu, -Trp, -His media and (2) SD/-Leu, -Trp, -Ade media.

As a result, the binding activity of each of the c-Met prey protein (DNA derived from A-G described above) was as follows:

| | <binding activity |
|---|---|
| | with MERMUC |
| <prey DNA> | bait protein> |
| A | + (bound) |
| B | + (bound) |
| C | − (no binding) |
| D | + (bound) |
| E | + (bound) |
| F | + (bound) |
| G | − (no binding). |

The 91 amino acids present at the C-terminus of the human c-Met β chain was revealed to bind to the C-terminal region of MERMUC (Example 1). The above results indicate that the deletion of 30 amino acid residues from the N-terminus or 40 amino acid residues from the C-terminus of the C-terminal 91 amino acids results in a loss of the binding ability to MERMUC.

The results revealed that the c-Met intramolecular region (binding site) exhibiting the binding ability to MERMUC is located within the common region of the above mentioned prey peptides. Namely, the binding site is located within the region consisting of about 40 amino acids on the N-terminal side from approximately the 30th amino acid from the C-terminus (from about amino acid 1013 to about amino acid 1052 of SEQ ID NO: 4; corresponding to SEQ ID NO: 5).

This common region contains 2 tyrosine residues (amino acids 1349 and 1356 of SEQ ID NO: 2; amino acids 1042 and 1049 of SEQ ID NO.4) that are autophosphorylated upon activation of the HGF receptor c-Met via the binding with HGF.

### EXAMPLE 6

### Identification of HGF Receptor c-Met β Chain Binding Region within Human MERMUC Molecule (Part 1)

The present examination was performed using the previously reported yeast two-hybrid system (references are described above) according to conventional method and the experimental protocols provided with the reagents.

Three bait vectors carrying DNAs corresponding to 3 MERMUC bait proteins described below were prepared by PCR based on the novel finding (Example 1) that the C-terminal region of human MERMUC cytoplasmic region binds to the C-terminal region of human c-Met:
A. The entire human MERMUC cytoplasmic region, i.e., DNA corresponding to the C-terminal 260 amino acids within the MERMUC cytoplasmic region (dubbed [C260]; amino acids 244-503 of SEQ ID NO: 10);
B. DNA corresponding to the region wherein 53 amino acids are deleted from the C-terminus of C260 (dubbed [C260A53]; amino acids 244-450 of SEQ ID NO: 10); and
C. DNA corresponding to the C-terminal 53 amino acids of C260 (dubbed [C53]; amino acids 451-503 of SEQ ID NO: 10).

PCR was performed using cDNA encoding the full-length human MERMUC (SEQ ID NO: 10) as a template and primer set as follows: primer for C260: DNAs described in SEQ ID NO: 27 and SEQ ID NO: 28, respectively;
primer for C260AC53: DNAs described in SEQ ID NO: 29 and SEQ ID NO: 30, respectively; and
primer for C53: DNAs described in SEQ ID NO: 31 and SEQ ID NO: 32, respectively.

Each of the obtained PCR product DNAs was inserted into plasmid pGBKT7 to produce a bait vector.

Yeast AH109 strain was cotransfected with each of the bait vectors and the prey vector carrying human HGF receptor c-Met (DNA corresponding to the C-terminal 91 amino acids of the human c-Met; clone No. 3-309) identified in Example 1.

The binding ability of MERMUC bait protein derived from each of the bait vectors to the HGF receptor c-Met β chain was analyzed by the yeast two-hybrid system described above.

As a result, both C260 (containing almost the entire MERMUC cytoplasmic region) and C53 (the C-terminal 53 amino acids of the MERMUC cytoplasmic region) possessed the binding activity to the HGF receptor c-MET β chain. On the other hand, C260AC53, wherein the C-terminal 53 amino acids of C260 were deleted, completely lost the binding activity to the HGF receptor c-Met β chain.

These results revealed that the C-terminal portion of MERMUC, particularly, the region comprising about 53 amino acids from the C-terminus (amino acids 451-503 of SEQ ID NO: 10) contribute to the binding of MERMUC to the HGF receptor c-Met.

Similar as above, the presence of binding between mouse MERMUC (amino acid/SEQ ID NO: 12; cDNA/SEQ ID NO:11) and mouse HGF receptor, as well as their binding sites were analyzed using the yeast two hybrid system.

cDNAs encoding each of the mouse MERMUC and mouse HGF receptor were prepared by cloning through the RT-PCR method from mouse kidney derived RNA.

As a result, the mouse MERMUC was revealed to bind at a region comprising about 53 amino acids of the C-terminus to the C-terminal region of the mouse HGF receptor β chain similar to the binding between human MERMUC and human c-Met. This observation explains the high amino acid sequence homology between human and mouse in the region comprising the C-terminal 53 amino acids of MERMUC. It also indicates that the C-terminal region is important for the binding to the HGF receptor regardless of species.

### EXAMPLE 7

### Identification of HGF Receptor c-Met β Chain Binding Region within Human MERMUC Molecule (Part 2)

The HGF receptor c-Met binding region revealed in the previous Example that exists in the human MERMUC C-terminal region (the region comprising the C-terminal 53 amino acids) was further analyzed in more detail. The analysis was performed by the yeast two-hybrid system similar to the previous Example.

Bait proteins corresponded to a part of the human MERMUC C-terminal region (randomly selected 10 continuous amino acids) as below:
A. amino acids 429-438 of SEQ ID NO.10 (overlaps 5 amino acids with B);
B. amino acids 434-443 of SEQ ID NO.10 (overlaps 5 amino acids with A and C, respectively);
C. amino acids 439-448 of SEQ ID NO.10 (overlaps 5 amino acids with B and D, respectively);
D. amino acids 444-453 of SEQ ID NO.10 (overlaps 5 amino acids with C and E, respectively);
E. amino acids 449-458 of SEQ ID NO.10 (overlaps 5 amino acids with D and F, respectively);
F. amino acids 454-463 of SEQ ID NO.10 (overlaps 5 amino acids with E and G, respectively);
G. amino acids 459-468 of SEQ ID NO.10 (overlaps 5 amino acids with F and H, respectively);
H. amino acids 464-473 of SEQ ID NO.10 (overlaps 5 amino acids with G and I, respectively);
I. amino acids 469-478 of SEQ ID NO.10 (overlaps 5 amino acids with H and J, respectively);
J. amino acids 474-483 of SEQ ID NO.10 (overlaps 5 amino acids with I and K, respectively);
K. amino acids 479-488 of SEQ ID NO.10 (overlaps 5 amino acids with J and L, respectively);
L. amino acids 484-493 of SEQ ID NO.10 (overlaps 5 amino acids with K and M, respectively);
M. amino acids 489-498 of SEQ ID NO.10 (overlaps 5 amino acids with L and N, respectively); and
N. amino acids 494-503 of SEQ ID NO.10 (overlaps 5 amino acids with M).

For each of the bait proteins described above (10 amino acids), sense strand DNA (an EcoRI site added at the 5′ end) encoding the protein and antisense strand DNA (a SalI added at the 5′ end) were synthesized, annealed, and then inserted into the EcoRI/SalI site of plasmid pGBKT7 to construct a bait vector.

Each of the bait vectors was cointroduced into yeast AH109 strain with human HGF receptor c-Met prey vector (comprising DNA encoding the C-terminal 91 amino acids of the c-Met; Example 1). The binding ability of each of the bait proteins (randomly selected 10 amino acids of the human MERMUC C-terminal region) to the C-terminal region of human c-Met β chain was analyzed by the yeast two-hybrid system similar to the aforementioned Example.

As a result, only those bait proteins relating to the C-terminal 53 amino acids that were identified to be important for the binding activity to the c-Met β chain in the previous Example possessed the binding activity to the C-terminal region of human c-Met β chain.

This test result further indicates that the C-terminal 53 amino acid region of human MERMUC has an extremely important role in the binding with the human HGF receptor c-Met.

Furthermore, the region conserved among human, mouse and rat within the amino acid sequence of the C-terminal region contains a leucine repeat structure wherein leucine appears at every 4 amino acid residues. This leucine repeat structure is suggested to have an important role for the binding of MERMUC and c-Met.

### EXAMPLE 8

### Construction of Assay System that Enables Evaluation of the Binding of MERMUC and HGF Receptor c-Met

β-Galactosidase (β-gal) is an enzyme widely distributed from bacteria to higher eukaryotes that degrades/synthesizes lactose. The biological activity of β-gal is exerted via a homotetramer wherein 4 monomer molecules of β-gal primary structure are associated together.

The β-gal of E. coli (lacZ gene) has been particularly studied well and applied as an experimental research tool.

Three mutants, β-galΔα, β-galΔμ and β-galΔω, have been reported in a certain E. coli strain (A indicates deletion; Proc. Natl. Acad. Sci. USA., 1996, Vol. 93, p.12423-12427; Proc. Natl. Acad. Sci. USA., 1997, Vol. 94, p.8405-8410).

β-galΔα lacks a part of the N-terminal sequence (amino acids 50-602 of GenBank Accession Nos. P00722) which is the active site of wild-type β-gal. β-galΔμ lacks a part of the sequence in the middle of wild-type β-gal (amino acids 50-602 of GenBank Accession Nos. P00722). β-galΔω lacks apart of the C-terminal sequence (amino acids 790-1024 of GenBank Accession Nos. P00722) which is necessary to form a homotetramer in wild-type ≢2-gal monomers.

Therefore, β-galΔα or β-galΔω alone naturally do not exhibit enzyme activity. Furthermore, without binding to each other (including encounter), even in the coexistence of β-galΔα and β-galΔω no β-gal activity is expressed.

However, when two proteins, [X] and [Y], that have affinity to each other are fused with β-galΔα and β-galΔω, respectively, and the produced [X-β-galΔα] and [Y-β-galΔω] coexist, β-gal activity due to the encounter of these two fusion proteins by the affinity between X and Y appears (Proc. Natl. Acad. Sci. USA., 1997, Vol. 94, p. 8405-8410).

Applying this theory, the binding ability of MERMUC and HGF receptor c-Met was evaluated by establishing an assay system that enables evaluation of the binding between the two molecules.

Specifically, the entire human HGF receptor c-Met or the C-terminal region thereof and the entire human MERMUC or the C-terminal region thereof were used as X and Y of X-β-galΔα and Y-β-galΔω, respectively. The binding between MERMUC and c-Met is evaluated by coexpressing these two fusion proteins in animal cells, such as CHO cells, via genetic recombination techniques and using the β-galactosidase activity expressed in the recombinant cells as an indicator.

In order to construct the present evaluation system, expression vectors expressing each fusion polypeptide (fusion protein) described below were prepared. The method to prepare each of the vectors will be explained below:
A. human MERMUC (full-length; full)/β-galΔω;
B. human MERMUC (full-length; full)/β-galΔα;
C. human MERMUC (53 amino acid deletion at the C-terminus; ΔC53)/β-galΔω;
D. human MERMUC (53 amino acid deletion at the C-terminus; ΔC53)/β-galΔα; and
E. human c-Met (C-terminal 91 amino acids alone; C91)/β-galΔα.
   <8-1> Isolation of β-galΔα Gene

β-galΔα lacks the N-terminal amino acids (amino acids 12-42 of GenBank Accession Nos. P00722) of the wild-type β-gal and E. coli DH5α strain is known to produce this mutant (Gene, 1992, Vol. 122, p. 231-232).

Thus, DNA encoding the target β-galΔα was cloned using DNA of E. coli DH5α strain as a template, and designing PCR forward primer (SEQ ID NO: 33; a XhoI site added at the 5′ side) and reverse primer (SEQ ID NO: 34; a SalI site added at the 5′ side) were designed based on the nucleotide sequences corresponding to the regions comprising the N-terminal methionine and the C-terminal stop codon of the wild-type β-gal.

The β-galΔα gene fragment amplified by PCR was digested with restriction enzymes XhoI and SalI, inserted into the XhoI and SalI site of pBluescript KS, and then was introduced into E. coli. The plasmid containing β-galΔα used in the following experiments was prepared from this transformant.

The amino acid sequence of Δ-galΔα is shown in SEQ ID NO: 16.

<8-2> Isolation of β-galΔω Gene

β-GalΔω lacks a part of the C-terminal sequence (amino acids 790-1024 of GenBank Accession Nos. P00722) that is required for the formation of homotetramer in the wild-type β-gal monomer molecules (Proc. Natl. Sci. USA., 1996, Vol. 93, p. 12423-12427).

Thus, DNA encoding β-galΔω was prepared by PCR amplification of the region corresponding to the sequence from N-terminal methionine to the amino acid 789 using wild-type β-gal gene from E. coli DH5 strain as a template.

The following primers were used for the PCR:
1. forward primer: SEQ ID NO: 35 (a XhoI site added at the 5′ side of ATG); and
2. reverse primer: SEQ ID NO: 36 (a SalI site added at the 5′ side of CGG).

The PCR product was digested with restriction enzymes XhoI and SalI to prepare a β-GalΔω gene fragment. The fragment was inserted into the XhoI and SalI sites of pBluescript KS and then introduced into E. coli. A plasmid containing β-galΔω gene used for following experiments was prepared from this transformant.

<8-3> Preparation of DNAs Encoding MERMUC (Full) and MERMUC (ΔC53) Used for Fusion with β-gal Mutant

In order to fuse the DNA encoding β-gal mutant to the DNA encoding human MERMUC (full) or human MERMUC (ΔC53) in-frame, both the stop codon of human MERMUC (full) DNA and the C-terminal codon of human MERMUC (ΔC53) were converted to a XhoI site by PCR using primers described below. DNA encoding the full-length human MERMUC was used as a template:
<human MERMUC (full)> (amplification from N-terminal Met to amino acid 503)
forward primer: SEQ ID NO: 37 (a HindIII site added at the 5′ side of ATG), and
reverse primer: SEQ ID NO: 38 (a XhoI site added at the 5′ side of GCC); and
<human MERMUC (AC53)> (amplification from N-terminal Met to amino acid 450)
forward primer: SEQ ID NO 39 (a HindIII site added at the 5′ side of ATG), and
reverse primer: SEQ ID NO: 40 (a XhoI site added at the 5′ side of TGC).

Each of the PCR products was digested with restriction enzymes HindIII and XhoI, inserted into the HindIII and XhoI site of pBluescript KS, and then introduced into E. coli. Each of the plasmids carrying the DNA encoding any one of the fusion proteins A to D described above was prepared from the transformed E. coli strain.

<8-4> Preparation of DNA Encoding Human c-Met (C91)

In order to fuse DNA encoding β-galΔα to DNA encoding human c-Met (C91) in-frame, a DNA corresponding to the C-terminal 91 amino acids of the human HGF receptor c-Met (amino acids 993-1083 of SEQ ID NO: 4) was prepared by PCR using primer set described below:
forward primer: SEQ ID NO: 41 (ATG and HindIII site added at the 5′ side of AGA); and
reverse primer: SEQ ID NO: 42 (XhoI site added at the 5′ side of TCA).

Each of the obtained PCR product was digested with restriction enzymes HindIII and XhoI, inserted into the HindIII and XhoI site of pBluescript KS, and then introduced into E. coli. Plasmids carrying DNA encoding the fusion proteins described above were prepared from the transformed E. coli strain.

<8-5> Preparation of Expression Vectors for Fusion Proteins (A, B, C, D and E)

A) Preparation of Human MERMUC (Full)/β-galΔω Expression Vector

Each of the plasmids prepared above was digested with restriction enzymes HindIII and XhoI or XhoI and SalI to cut out DNA fragments encoding human MERMUC (full) and β-galΔω, respectively. The obtained two DNA fragments were ligated at the XhoI site, and then inserted into the HindIII/SalI site of pcDNA3 vector to construct the objective human MERMUC (full)/β-galΔω expression vector.

B) Preparation of Human MERMUC (Full)/β-galΔω Expression Vector

Each of the plasmids produced above was digested with restriction enzymes HindIII and XhoI or XhoI and SalI to cut out DNA fragments encoding human MERMUC (full) and β-galΔα, respectively. The obtained two DNA fragments were ligated at the XhoI site and then inserted into the HindIII/SalI site of pcDNA3 vector to construct the objective human MERMUC (full)/Δ-galΔα expression vector.

C) Preparation of Human MERMUC (AC53)/β-galΔω Expression Vector

Each of the plasmids produced above was digested with restriction enzymes HindIII and XhoI or XhoI and SalI to cut out DNA fragments encoding human MERMUC (ΔC53) and Δ-galΔω, respectively. The obtained two DNA fragments were ligated at the XhoI site and then inserted into the HindIII/SalI site of pcDNA3 vector to construct the objective human MERMUC (ΔC53)/β-galΔω expression vector.

D) Preparation of Human MERMUC (ΔC53)/β-galΔα Expression Vector

Each of the plasmids produced above was digested with restriction enzymes HindIII and XhoI or XhoI and SalI to cut out DNA fragments encoding human MERMUC (ΔC53) and β-galΔα, respectively. The obtained two DNA fragments were ligated at the XhoI site and then inserted into the HindIII/SalI site of pcDNA3 vector to construct the objective human MERMUC (AC53)/β-galΔα expression vector.

E) Preparation of Human c-Met (C91)β-galΔα Expression Vector

Each of the plasmids produced above was digested with restriction enzymes HindIII and XhoI or XhoI and SalI to cut out DNA fragments encoding human c-Met (C91) and Δ-galΔα, respectively. The obtained two DNA fragments were ligated at the XhoI site and then inserted into the HindIII/SalI site of pcDNA3 vector to construct the objective human c-Met (C91)/βgalΔα expression vector.

<8-6> Assay to Evaluate the Binding of MERMUC with HGF Receptor c-Met

CHO cells were cotransfected with a mixture containing the above-described 5 fusion protein expression vectors in combination described below at equivalent amounts using commercially available transfection reagent (GeneJammer Transfection Reagent; STRATAGENE) according to the experimental protocol:
I) A+E, i.e., MERMUC (full)/β-galΔω+c-Met (C91)/β-galΔα;
II) A+B, i.e., MERMUC (full)/β-galΔω+MERMUC (full))/β-galΔα;
III) C+E, i.e., MERMUC (ΔC53)/β-galΔω+c-Met (C91)/β-galΔα; and
IV) C+D, i.e., MERMUC (ΔC53)/β-galΔω+MERMUC (ΔC53)/β-)/Δ-galΔα.

After incubating the cells for 48 hr at 37° C., β-Galactosidase activity in each cell was measured according to the experimental protocol using GAL-SCREEN SYSTEM (Applied Biosystems).

The results are shown in FIG. 6 and FIG. 7.

As a result, the combinations of (I), (II) and (III) showed a significantly higher β-galactosidase activity compared with cells transformed with the control (mock-control) vector alone. On the other hand, the combination of (III) did not show any activity.

These results confirm the following findings that were clarified by the Examples described above:
1. MERMUC binds to HGF receptor c-Met;
2. the c-Met binding region of MERMUC exists in the region containing about 53 amino acids of the C-terminal cytoplasmic region of MERMUC; and
3. the MERMUC binding region of c-Met exists in the region containing about 91 amino acids of the C-terminal cytoplasmic region of c-Met β chain.

Furthermore, the test result of the combination (IV) described above indicated that the site contributing to the binding among MERMUC molecules exists much near to the N-terminus than the C-terminal 53 amino acids that is the c-Met binding region of MERMUC.

Moreover, the above results indicate that substrates inhibiting the binding of MERMUC and c-Met, and substances that inhibit the intermolecular binding of MERMUC can be identified by the assay system constructed in the present test. Similarly, the use of the assay system was indicated to enable measurement and evaluation of the existence or degree of activity of a substance to inhibit the binding between MERMUC and c-Met, or to suppress or inhibit the intermolecular binding of MERMUC.

### EXAMPLE 9

### Biochemical Analysis of Binding of HGF Receptor c-Met and MERMUC

The C-terminal region of the HGF receptor c-Met β-chain where MERMUC binds is known as the multifunctional docking site that contains autophosphorylated tyrosine residues. This region is extremely important for transmission of signals into the cell that are generated by the binding of HGF to HGF receptor c-Met. Therefore, HGF signaling was predicted to be physiologically modulated by the binding of MERMUC molecules to this region.

Thus, to examine the physiological importance of the binding of MERMUC and HGF receptor c-Met, following experiments were performed using the MERMUC expressing recombinant cells prepared in the above-described Example in which cells the gene expression is regulated by Dox.

<9-1> Regulation of MAP Kinase Activation via HGF Receptor

According to previous studies, the activation of MAP kinase caused by HGF signal transduction is revealed to be accomplished through a stepwise process as below (Cell., 1994, Vol. 77, p. 261-271; Experimental Medicine, 1997, Vol. 15, p. 1033-1039; Nature, 1998, Vol. 391, p. 285-288):
(1) homodimerizaion of HGF receptor c-Met upon binding of HGF to the HGF receptor c-Met;
(2) autophosphorylation of tyrosine residues in the C-terminal region of C-Met β chain;
(3) recruitment of growth factor receptor-bound protein-2 (Grb2) to the phosphorylation site;
(4) Ras activation; and
(5) mitogen-activated protein (MAP) kinase activation.

Therefore, the existence of physiological effect of MERMUC expression on intracellular MAP kinase activation induced by HGF stimulation was analyzed.

Specifically, MERMUC non-producing system and MERMUC producing system were prepared by culturing the above-mentioned MERMUC/293 cell strain in the presence or absence of Dox, respectively. The degree of MAP kinase activation elicited immediately after the addition of HGF into each of the system was compared.

Each of the cells (MERMUC non-producing system) wherein MERMUC expression is suppressed by culturing in the presence of Dox and cells (MERMUC producing system) wherein MERMUC expression is induced by culturing in the absence of Dox was stimulated with human recombinant HGF (#375228; CALBIOCHEM). Immediately thereafter, cell lysate was prepared for the evaluation of MAP kinase activity.

The cell lysate was separated by SDS polyacrylamide gel electrophoresis (SDS-PAGE), and then subjected to western blotting according to conventional method using anti-MAP kinase polyclonal antibody (p44/42 MAP kinase antibody, #9102; Cell Signaling Technology) and anti-phosphorylated MAP kinase polyclonal antibody (Phospho-p44/42 MAP kinase (Thr202/Thr204) antibody, #9101L; Cell Signaling Technology).

The amount of MAP kinase protein in both of the samples was confirmed to be the same using the former antibody described above. Then, the signal intensities of phosphorylated MAP kinase that are detected with the latter antibody described above were compared.

The results are shown in FIG. 8.

As a result, the MAP kinase activity was significantly reduced in MERMUC producing cells compared with MERMUC non-producing cells.

Furthermore, MAP kinase activity gradually decreased in response to the increase of MERMUC production due to the lowering of the Dox concentration.

These results revealed that HGF signal transduction is suppressed depending on the amount of MERMUC production and that MERMUC down-regulates HGF signal transduction via the binding to the HGF receptor.

<9-2> Regulation of Autophosphorylation in Cytoplasmic Region of HGF Receptor c-Met

The decrease in MERMUC production-dependent MAP kinase activity revealed in the above Example was suggested to be caused by a decrease in autophosphorylation of the cytoplasmic region of the HGF receptor c-Met during the HGF signal transduction process.

Therefore, the effect of MERMUC production on HGF signal transduction was examined by analyzing the level of autophosphorylation of HGF receptor that is induced by adding HGF to MERMUC overproducing cells and MERMUC non-producing cells.

Cell lysate was prepared from MERMUC overexpressing cells and MERMUC non-producing cells that were obtained by culturing MERMUC/293 cells in the absence or presence of Dox, respectively, as described in the previous Example.

Immunoprecipitation treatment was performed by adding anti-HGF receptor polyclonal antibody to each of the cell lysate. Then, after separating the precipitated proteins by SDS-PAGE, Western blotting was performed using anti-phosphorylated tyrosine monoclonal antibody (Phospho-Tyrosine monoclonal antibody (P-Tyr-100), #9411; Cell Signaling Technology).

The amount of HGF receptor protein in both of the cell lysates was confirmed to be identical using the former antibody described above. Then, autophosphorylation of tyrosine residues in the cytoplasmic region of the HGF receptor c-Met β-chain was analyzed by comparing the signal intensities detected with the latter antibody described above.

The results are shown in FIG. 9.

As a result, no difference in tyrosine residue phosphorylation could be observed between the two cells. Therefore, the results indicate that MERMUC production and the binding of MERMUC to the HGF receptor c-Met donot affect the autophosphorylation of tyrosine residues in the cytoplasmic region of the HGF receptor that is induced by HGF stimulation.

<9-3> Regulation of Grb2 Binding to Phosphorylated Tyrosine in Cytoplasmic Region of HGF Receptor

The influence of MERMUC production (namely, the binding of MERMUC and c-Met) on a step of HGF signal transduction process, i.e., the binding of Grb2 molecule to phosphorylated tyrosine in the cytoplasmic region of the HGF receptor c-Met β chain was examined.

The amount of Grb2 bound to phosphorylated tyrosine was evaluated as the amount of coprecipitated HGF receptor protein in the immunoprecipitation of Grb2 with anti-Grb2 antibody.

Cell lysate was prepared from both the MERMUC over expressing cells and MERMUC non-producing cells that were obtained by incubating MERMUC/293 cells in the absence or presence of Dox, respectively, as described in the previous Example.

Grb2 protein was immunoprecipitated according to conventional method by adding anti-Grb2 polyclonal antibody (GRB2 (C-23): sc-255; Santa Cruz Biotechnology) into each of the lysate. Next, after separating HGF receptor protein that coprecipitated with Grb2 protein by SDS-PAGE, Western blotting was performed using anti-HGF receptor polyclonal antibody.

The amount of HGF receptor protein coprecipitated with Grb2 was evaluated based on the detected signal intensity of HGF receptor.

Furthermore, as a control experiment, Gab1 that is also known to be recruited to the phosphorylated tyrosine site in the cytoplasmic region of the HGF receptor c-Met, similar to Grb2, was analyzed in the same way as described above. Anti-Gab1 polyclonal antibody (Gab1 (H-198): sc-9049; Santa Cruz Biotechnology) was used for the control experiment.

The results are shown in FIG. 10.

As a result, the amount of HGF receptor c-Met that coprecipitated with Grb2 was significantly decreased in MERMUC producing cells compared with MERMUC non-producing cells. On the other hand, no significant difference could be observed in the amount of c-Met that coprecipitates with Gab1 between MERMUC non-producing cells and MERMUC producing cells.

These results indicate that the binding of Grb2 to phosphorylated tyrosine in the cytoplasmic region of the HGF receptor c-Met β chain is inhibited by MERMUC production.

<9-4> Regulation of MMP-1 and MMP-9 Gene Expressions

Expression of matrixmetalloprotease-1 (MMP-1) and matrixmetalloprotease-9 (MMP-9) is known to be induced upon HGF stimulation in cultured cell strains and kidney tissues (Kidney Int., 2000, Vol. 58, p.2028-2043; Carcinogenesis, 2000, Vol. 21, p. 1079-1085).

Therefore, human MERMUC expressing recombinant cells (MERMUC/293 cells; as described in the previous Example) were produced from HEK293 cells established from human kidney derived cells as a host, and the influence of MERMUC production on MMP-1 and MMP-9 expression induced through HGF stimulation in the cells was analyzed by RT-PCR.

MERMUC overexpressing cells and MERMUC non-producing cells were prepared by incubating MERMUC/293 cells in the absence or presence of Dox, respectively, similarly as described in the previous Example.

Human HGF was added to each of the cells and RNAs were prepared from each of the cells immediately after HGF addition (time 0) and 3, 6 and 24 hr after HGF addition according to conventional method. Each of the RNA sample was used as a template and RT-PCR was performed according to conventional method using a primer set described below to determine the mRNA expression level of MMP-1, MMP-9 and GADH (control) in each of the samples:
primers for MMP-1: SEQ ID NO: 43 and SEQ ID NO: 44;
primers for MMP-9: SEQ ID NO: 45 and SEQ ID NO: 46; and
primers for GAPDH: SEQ ID NO: 47 and SEQ ID NO: 47.

The results are shown in FIG. 11.

Increase over time in MMP-1 mRNA expression was observed in MERMUC non-producing cells. However, the expression induction was significantly suppressed in MERMUC producing cells.

Moreover, a peak was observed in the expression induction of MMP-9 mRNA 3 hr after HGF stimulation in MERMUC non-producing cells. However, almost no expression induction of MMP-9 mRNA was observed in MERMUC producing cells.

These results indicate that the expression of both MMP-1 and MMP-9 genes induced by HGF stimulation is decreased due to the overexpression of MERMUC protein, and that the decrease in the expression of both of the genes result from the decreased MAP kinase activation due to the down-regulation of HGF signaling by MERMUC protein.

The findings described below were identified in relation to the MERMUC function and the down-regulation mechanism of HGF signaling from the test results mentioned above:
1) MERMUC binds in the vicinity of autophosphorylated tyrosine in the cytoplasmic region of the HGF receptor c-Met β chain, and thereby, inhibits the binding of Grb2 protein that is recruited to this site;
2) as a result, MAP kinase activity that should be elicited through the signal transduction due to the binding of HGF/HGF receptor c-Met is decreased;
3) as a result of the decrease in MAP kinase activity, biological reactions thereafter (for example, the expression of MMP-1, MMP-9 and such) are inhibited; and
4) finally, HGF/HGF receptor c-Met signaling is down-regulated.

### EXAMPLE 10

### Production and Analysis of MERMUC Overexpressing Transgenic Mice

<10-1> Production of MERMUC-Tg Mouse

The proliferation of primary cultured cells prepared from liver is reported to be enhanced by HGF stimulation (Proc. Natl. Acad. Sci. USA., 1986, Vol. 83, p. 6489-6493). Utilizing this phenomenon, the effect of the increase in MERMUC protein production in liver cells on HGF stimulation-dependent cell proliferation was analyzed using liver cells of MERMUC overexpressing transgenic (Tg) mouse (hereafter, simply referred to as MERMUC-Tg mouse, MERMUC-Tg or Tg).

MERMUC-Tg mouse was produced according to conventional method (“Manipulating the mouse embryo, a laboratory manual”, 1989, Chap. 2-4, p.77-203, Kindai Shuppan; “The latest Laboratory Manual for Animal Cells”, Chap 7, p361-408, 1990; LIC Press).

cDNA (SEQ ID NO: 11) encoding the full-length mouse MERMUC was inserted into expression vector PCAGGS (Gene, 1991, Vol. 108, p193-200) carrying the avian β-actin promoter using DNA blunt end kit (TAKARA) to obtain a mouse MERMUC expression vector. The vector was linearized by digestion with restriction enzyme and was used as the mouse MERMUC gene for Tg production.

A white ICR mouse (female; Charles River Japan) and a vasoligated white ICR mouse (male; Charles River Japan) were crossed to obtain a female ICR mouse possessing a plug (or a vaginal gland) as the surrogate mouse. Furthermore, mouse for harvesting fertilized eggs to introduce the mouse MERMUC expression vector was produced by crossing an overovulation ICR mouse (female; Charles River Japan) prepared by administration of PMS (5 unit; Sigma) and hCG (5 unit; Sigma) with an ICR mouse (male; Charles River Japan). After mating, the oviduct was extirpated from the ICR mouse (female), treated with hyaluronidase to obtain only fertilized eggs, and the eggs were preserved in culture media.

The introduction of mouse MERMUC gene into a fertilized egg was performed according to conventional method using a manipulator under a microscope. The fertilized egg was fixed with a retention needle, and a solution containing the mouse MERMUC gene diluted with Tris-EDTA buffer was injected into the male pronucleus of the fertilized egg using a DNA injection needle at 37° C.

After gene transfer, fertilized eggs keeping normal conditions were selected as the mouse MERMUC gene transferred eggs and inserted into the fimbriae of uterine tube in the ovary of the surrogate mouse (white ICR mouse). Mouse off springs (chimera mice) born from the surrogate mother were obtained as MERMUC-Tg mice.

Northern blot analysis was performed according to conventional method using mRNA extracted from each of various organs (liver, kidney, heart, lung, brain, etc.) of the produced MERMUC-Tg and normal mice (non-Tg mice; normal mice born from the above-mentioned surrogate mother) to analyze the mRNA expression level of mouse MERMUC.

As a result, mRNA expression of mouse MERMUC in Tg mice was significantly higher in all of the organs compared to the non-Tg mice. Thus, the produced MERMUC-Tg mice were confirmed to be mouse MERMUC overexpressing mice.

<10-2> Analysis of the Effect of Increase in MERMUC Production On HGF Stimulation-Dependent Liver Cell Proliferation

Primary cell culture of hepatocytes from liver cells obtained from the liver of each of the MERMUC-Tg mice (8 weeks old, 4 mice) and non-Tg mice (8 weeks old, 4 mice) was prepared by collagenase treatment according to the method of Berry and Friend, and Seglen et al. (Berry & Friend, J. Cell Biol., 1969, Vol. 43, p. 506-520; Seglen et al., Methods in Cell Biol., 1976, Vol. 13, p. 29-83).

Each of the obtained hepatocytes (2×10³ cells) was cultured in serum-free media for 15 hr, HGF (0, 1 or 3 ng/ml) was added to the media to induce HGF-dependent cell proliferation, and was further cultured. Similarly, cultivation was performed by adding epidermal growth factor (EGF; 0, 1, or 10 ng/ml) in place of HGF to the culture as a control.

After 24 hr from HGF addition, cell numbers were counted using Cell Count Reagent SF (Nacalai tesque) according to the experimental protocol provided with the reagent to calculate the relative cell proliferation rate after HGF (or EGF) addition. Tests were conducted in triplicate.

The results are shown in FIG. 12.

There was no difference between MERMUC-Tg and non-Tg in the control EGF stimulated hepatocyte proliferation, and both showed dosage-dependent cell proliferation (FIG. 12-B).

On the other hand, the proliferation due to HGF stimulation of hepatocytes derived from MERMUC-Tg mice showed a significant decrease in the proliferation (reactivity) compared with the cells from non-Tg mice (FIG. 12-A).

<10-3> Analysis of the Effect of Increase in MERMUC Production On Activation of Intracellular MAP Kinase

The changes in intracellular MAP kinase activation after HGF stimulation in hepatocytes derived from each of the MERMUC-Tg mice and non-Tg mice were analyzed similarly as in Example 9.

The results are shown in FIG. 13.

The results showed a significant decrease in MAP kinase activation in MERMUC-Tg derived cells.

These results indicate that the proliferation of various cells, including liver cells, is suppressed through the suppression of functional expression of HGF/HGF receptor by the binding of MERMUC to the HGF receptor. They also indicate down-regulation of cell proliferation, and regeneration and neogenesis of tissues, organs, blood vessels and such via the suppression of functional expression of HGF/HGF receptor by MERMUC.

### EXAMPLE 11

### Analysis of Increase in MERMUC Binding Affinity with HGF Receptor Due to MERMUC Self-Association

The yeast two-hybrid system and the immunoprecipitation method described above showed that MERMUC forms multimers by self-association.

In the present experiment, the relationship between MERMUC self-association and the binding between MERMUC and HGF receptor was analyzed by establishing an evaluation system that reproduces the stimulation-dependent MERMUC self-association.

<11-1> Establishment of Evaluation System Reproducing Stimulation-Dependent MERMUC Self-Association

The following three expression vectors were constructed by the method described below:
(1) plasmid (hereafter, referred to as “mFas-MERMUC”) expressing a fusion chimeric protein consisting of a part of mouse Fas (CD95) (consisting of the extracellular region and transmembrane region) and the cytoplasmic region of human MERMUC;
(2) plasmid (hereafter, referred to as “mFas-MERMUC-β-galΔω”) expressing a fusion chimeric protein consisting of a part of mouse Fas (consisting of the extracellular region and transmembrane region), the cytoplasmic region of human MERMUC and β-galΔω described above; and
(3) plasmid (hereafter, referred to as “mFas-MERMUC-β-galΔα”) expressing a fusion chimeric protein consisting of a part of mouse Fas (consisting of the extracellular region and transmembrane region), the cytoplasmic region of human MERMUC and β-galΔα described above.

The mouse Fas described above was as reported (GenBank Accession Nos. P 25446 and NP 032013; J. Immunol., 1992, Vol. 148, No. 4, p. 1274-1279; etc.).

cDNA encoding the part of mouse Fas (the extracellular region and transmembrane region) was prepared by PCR using mouse spleen cDNA library (CLONTECH) as a template and a primer set as follows:
forward primer: SEQ ID NO: 49; and
reverse primer: SEQ ID NO: 50.

cDNA encoding the cytoplasmic region of human MERMUC was prepared by PCR using full-length human MERMUC cDNA (SEQ ID NO: 9) described above as a template and a primer set as follows:
forward primer: SEQ ID NO: 51; and
reverse primer: SEQ ID NO: 52.

Each of the obtained cDNA was subcloned into pTargeT vector (Promega) and then both fragments were obtained by redigestion with restriction enzymes XbaI and NotI. The digested fragments were ligated at the XbaI site and inserted into pcDNA3 vector to obtain mFas-MERMUC expression plasmid.

Expression plasmids for mFas-MERMUC-β-galΔω and mFas-MERMUC-β-galΔα were constructed as follows.

Both of the above-prepared human MERMUC (full-length)/β-galΔα and human MERMUC (full-length)/β-galΔω were digested with ApaI to prepare cDNAs encoding MERMUC (cytoplasmic region)-β-galΔα and MERMUC (cytoplasmic region)-β-galΔω. Expression plasmids for mFas-MERMUC-β-galΔω and mFas-MERMUC-β-galΔα were prepared by inserting the obtained respective cDNAs downstream of the ApaI site of the previously constructed mFas-MERMUC.

Next, expression plasmids for mFas-MERMUC-β-galΔω and mFas-MERMUC-β-galΔω were added to HEK293 cells and cultured at 37° C. for cotransfection using GeneJammer Transfection Reagent (STRATAGENE) according to the attached protocol. Recombinant cells coexpressing mFas-MERMUC-β-galΔω and mFas-MERMUC-β-galΔα on the cell membrane were obtained.

Both the bound and unbound states of the extracellular Fas region can be created by treating this recombinant cell with anti-Fas antibody that either possess or lacks the activity to induce the association of Fas molecules. Furthermore, the association (binding) of MERMUC protein portion within the cells induced by the association of the Fas region can be evaluated by measuring β-galactosidase activity.

Forty eight hr after the transfection of both of the expression plasmids into the recombinant cells, the cells were treated with anti-Fas antibody and negative control antibody (1 or 10 μg/ml each) for 3 hr, respectively, and then, β-galactsidase activity in each of the cells was measured using Gal-Screen System (Applied Biosystems) according to the accompanying protocol.

Hamster anti-mouse Fas antibody (PK-8; Medical and Biological Laboratories) that has the activity to induce Fas molecule association or rat anti-mouse Fas antibody (RMF6; Medical and Biological Laboratories) that does not have the activity was used as the anti-Fas antibody. On the other hand, hamster antibody or rat antibody that does not react with mFas was used as the negative control antibody.

The results are shown in FIG. 14 As a result, no change in β-galactosidase activity was detected when recombinant cells that coexpress mFas-MERMUC-β-galΔω and mFas-MERMUC-β-galΔα were treated with anti-Fas antibody lacking Fas molecule association inducing activity. However, the treatment with anti-Fas antibody that has Fas molecule association inducing ability increased the β-galactosidase activity in a concentration-dependent manner.

These results indicate that, using the recombinant cells that coexpress mFas-MERMUC-β-galΔω and mFas-MERMUC-β-galΔα produced in the present Example, the intracellular association of MERMUC region can be freely regulated by regulating the association of extracellular Fas protein region with anti-Fas antibody.

<11-2> Analysis of the Effect of MERMUC Self-Association on Binding Affinity of MERMUC and HGF Receptor

The effect of self-association of MERMUC molecules on the binding affinity of MERMUC and HGF receptor c-Met was analyzed by immunoprecipitation of MERMUC and HGF receptor (immunocoprecipitation) using the recombinant cells produced above.

mFas-MERMUC expressing recombinant cells were prepared by transfecting HEK293 cells with mFas-MERMUC expression plasmid produced above. The cells were treated with each of the above-described anti-Fas antibody and then cell lysate was prepared by lysing cells according to conventional method. Commercially available anti-HGF receptor c-Met polyclonal antibody h-Met (C-28) (sc-161; Santa Cruz Biotechnology) described above was added to the cell lysate, reacted with protein-G Sepharose, and then HGF receptor protein/anti-c-Met antibody complex and protein components bound to the complex were precipitated by centrifugation.

The precipitate was dissolved in electrophoresis buffer, separated by SDS-PAGE, and then subjected to Western blotting according to convention method using anti-mouse Fas antibody (R&D) to evaluate the amount of coprecipitated mFas-MERMUC protein.

The results are shown in FIG. 15.

The results indicate significant increase of coimmunoprecipitated mFas-MERUMUC in cells treated with anti-Fas antibody (RK-8) that possesses Fas molecule association inducing activity but not in cells treated with anti-Fas antibody (RMF6) lacking the association inducing activity or control antibody (non-reactive to mouse Fas; described above).

These results showed that the self-association (within the cytoplasmic region) of MERMUC promoted the binding ability of MERMUC with HGF receptor.

### EXAMPLE 12

### Analysis of Decrease in Binding of MERMUC with HGF Receptor by Suppression of MERMUC Self-Association

As described in the previous Example, the self-association ability of MERMUC is maintained even by the deletion of the C-terminal 53 amino acids. Therefore, the responsible region for MERMUC self-association was predicted to exist in the N-terminal side of the HGF receptor-binding region (the C-terminal 53 amino acid residues of MERMUC) of MERMUC.

To validate this prediction and at the same time synthesize a MERMUCMERMUC peptide that is able to suppress MERMUC self-association through the binding to the MERMUC self-association region, the following 4 types of peptides each corresponding to a part of MERMUC cytoplasmic region were synthesized by the method described later:
del-1: amino acid sequence from amino acids 244 to 345 of SEQ ID NO: 10;
del-2: amino acid sequence from amino acids 244 to 380 of SEQ ID NO: 10;
del-3: amino acid sequence from amino acids 244 to 415 of SEQ ID NO: 10; and
del-4: amino acid sequence from amino acids 244 to 450 of SEQ ID NO: 10.

cDNAs encoding each of the human MERMUC cytoplasmic region fragments (del-1 to del-4) were prepared using full-length human MERMUC cDNA (SEQ ID NO: 9) as a template and a primer set as follows:

(1) del-1
forward primer: SEQ ID NO: 53 and
reverse primer: SEQ ID NO: 54;

(2) del-2
forward primer: SEQ ID NO: 53 and
reverse primer: SEQ ID NO: 55;

(3) del-3
forward primer: SEQ ID NO: 53 and
reverse primer: SEQ ID NO: 56; and

(4) del-4
forward primer: SEQ ID NO: 53 and
reverse primer: SEQ ID NO: 57.

Furthermore, ATG corresponding to a methionine codon and a HindIII site to the 5′ side thereto was added at the 5′ side of the forward primer (SEQ ID NO: 53). Moreover, TTA corresponding to the antisense of a stop codon and a XhoI site to the 5′ side thereto was added at the 5′ side of each reverse primer.

Each of the cDNA obtained by PCR was digested with HindIII and XhoI and inserted into the HindIII/XhoI site of pcDNA3 vector to obtain an expression vector expressing each of the human MERMUC cytoplasmic region fragments (del-1 to del-4).

Next, the following combination of recombinant protein expression vectors were added to CHO-K1 cells and cultured at 37° C. for 48 hr for cotransfection using GeneJammer Transfection Reagent (STRATAGENE):
1) vector expressing MERMUC (full)/β-galΔω (Example 8)), vector expressing c-Met (C91)/β-galΔα (Example 8), and vector expressing any one of del-1 to del-4 (described above); or
2) vector expressing MERMUC (full)/β-galΔω (Example 8)), vector expressing MERMUC (full)/β-galΔα (Example 8), and vector expressing any one of del-1 to del-4 (described above).

Intracellular β-Galactosidase activity of the recombinant cells was measured using Gal-Screen System (Applied Biosystems) according to the accompanying protocol 48 hr after plasmid transfection.

As a control, recombinant cells transfected with pcDNA3 vector (called “mock”) instead of vectors expressing one of del-1 to del-4 were used in above (1) and (2), respectively.

The results are shown in FIG. 16 to FIG. 18.

As shown in FIG. 16, del-4 showed the strongest suppression on MERMUC self-association. Furthermore, the greatest decrease in the binding of MERMUC and HGF receptor c-Met was achieved by del-4 as shown in FIG. 17. Moreover, the suppression of MERMUC self-association and the suppression of the binding of MERMUC and HGF receptor c-Met by del-4 both increased in del-4 dosage-dependent manner.

### EXAMPLE 13

### Analysis of Recovery of MAP Kinase Activation Due to Suppression of Binding of MERMUC and HGF Receptor

As described in the previous Example, HGF receptor mediated MAP kinase activation due to HGF stimulation was decreased by the binding of MERMUC to the HGF receptor.

Therefore, it was examined whether the decrease in MAP kinase activity is suppressed by the MERMUC cytoplasmic region fragment del-4 described above.

The del-4 protein expression vector produced above was added to Tet-Off/MERMUC expressing recombinant cells (MERMUC/293 cells) (produced in Example 2) and cultured at 37° C. for 48 hr for transfection using GeneJammer Transfection Reagent (STRATAGENE) according to the accompanying protocol.

Cells were similarly transfected with pcDNA3 vector [mock] that lack the cDNA encoding del-4 instead of the del-4 expression vector as a control.

The cells were stimulated with human recombinant HGF (CALBIOCHEM), and cell lysate was prepared according to conventional method immediately after the stimulation to evaluate the activation level of MAP kinase (also referred to as Erk).

The cell lysate was separated by SDS-PAGE, and then subjected to Western blotting according to conventional method using anti-phosphorylated MAP kinase polyclonal antibody (Phospho-p44/42 MAP kinase (Thr202/Thr204) antibody; Cell Signaling Technology) to determine the MAP kinase activation level based on the obtained signal intensity.

The results are shown in FIG. 19.

The results showed, as expected, decrease in the phosphorylated MAP kinase level after HGF stimulation due to the expression of MERMUC protein in control cells transfected with the mock vector (pcDNA3). However, the suppression was removed in the del-4 expressing cells and a significant level of phosphorylated MAP kinase was observed.

These results indicate a close relationship between MERMUC self-association (multimerization) and the binding ability of MERMUC with the HGF receptor. Furthermore, it was suggested that the increase in MERMUC self-association enhances the binding ability (binding affinity) of MERMUC with the HGF receptor, and as a result, enhances the ability of HGF to suppress the HGF receptor mediated MAP kinase activity.

### EXAMPLE 14

### Analysis of Induction of MERMUC Protein Phosphorylation by HGF Stimulation

The intracellular signal transduction via the HGF receptor after HGF stimulation is modulated through the binding of MERMUC to the HGF receptor c-Met. Therefore, it was predicted that MERMUC itself was also receive some kind of modification/regulation (for example, feedback regulation) downstream of HGF/HGF receptor signaling.

The human MERMUC cytoplasmic region contains many amino acid residues, i.e., 45, 37 and 1 threonine, serine and tyrosine residues, respectively, that may be phosphorylated. Therefore, actual phosphorylation of the residues was analyzed as described below.

After labeling the Tet-Off/MERMUC expressing recombinant cells (MERMUC/293 cells) produced above with (³²P] orthophosphate, the cells were stimulated with human recombinant HGF (20ng/ml; 5 min). Next, cell lysate was prepared by lysing cells according to conventional method. Anti-human MERMUC polyclonal antibody prepared as above (Example 2) was added to and reacted with the obtained cell lysate, bound to protein-G Sepharose, and then MERMUC protein and anti-MERMUC polyclonal antibody complex was precipitated by centrifugation.

The precipitate was extracted with 1% SDS and similar immunoprecipitation procedure was performed to reduce the background. The sample prepared by this procedure was separated by SDS-PAGE and analyzed by autoradiography.

Furthermore, MERMUC expression in the above mentioned recombinant cells was confirmed to be constant regardless of the presence or absence of HGF by Western blotting according to conventional method using anti-human MERMUC polyclonal antibody.

The results are shown in FIG. 20.

As a result, a strong signal band indicating phosphorylated MERMUC protein was detected in cells wherein MERMUC was overexpressed by cultivation without Dox via HGF stimulation.

The following experiments were conducted similarly as described above:
1) epidermal growth factor (EGF) was used in place of HGF;
2) MERMUC expressing cells were treated with HGF signaling suppressor, PI3 kinase inhibitor (LY294002; 10 μM; CALBIOCHEM), before HGF stimulation;
3) MERMUC expressing cells were treated with HGF signaling suppressor, MEK 1/2 inhibitor (U0126; 10 μM; CALBIOCHEM), before HGF stimulation;
4) MERMUC expressing cells were treated with PI3 kinase inhibitor (LY294002; 10 μM) before EGF stimulation; and
5) MERMUC expressing cells were treated with MEK 1/2 inhibitor (U0126; 10 μM) before EGF stimulation.

The results are shown in FIG. 21.

As a result, other growth factors such as EGF did not show effect to increase phosphorylation of MERMUC. Furthermore, the treatment of cells with PI3 kinase inhibitor and MEK 1/2 inhibitor revealed that the enhancement of MERMUC phosphorylation is suppressed only in the cells treated with PI3 kinase inhibitor.

Thus, MERMUC phosphorylation was supposed to be mediated by the PI3 kinase and Akt kinase pathway.

### EXAMPLE 13

### Analysis of Suppression on HGF-Dependent Liver Enlargement in MERMUC Overexpressing Transgenic Mice

It has been reported that HGF is transiently overexpressed in vivo by the injection of HGF expression plasmid DNA into the tail vein of normal mice, causing liver enlargement due to the enhancement of hepatocyte proliferation (Hepatology, 2001, Vol. 33, p848-859).

Therefore, to validate the down-regulation activity of MERMUC on HGF receptor mediated signaling, the method in the above mentioned report was applied to MERMUC overexpressing transgenic mice (MERMUC-Tg; 7 weeks old; male; 23-33 g) produced in the previous Example, and the effect due to the expression of MERMUC on liver enlargement induced by HGF overexpression was examined.

Human HGF expression plasmid (human HGF-cDNA in pcDNA3; Invitrogen) or plasmid pcDNA3 (mock) was injected into the tail vein of each of MERMUC-Tg mouse and littermate non-Tg mouse at a concentration of 28 μg/mouse (2.3 ml saline) within 5 to 10 seconds. The HGF level in serum of each of the mice was measured using a human HGF ELISA kit (Genzyme Techne) two days after the plasmid injection. The level of HGF production due to the administered plasmid in each animal was evaluated together with the changes in liver weight upon laparotomy.

The results are shown in FIG. 22.

As a result, the ratio of liver weight to body weight increased about 1.6 times due to the transient overexpression of HGF in non-Tg mice. However, the increase was suppressed to about 1.2 times in MERMUC-Tg mice. That is, significant difference in HGF dependent-liver enlargement was observed between Tg/non-Tg mice.

The findings hitherto forecast HGF-dependent liver enlargement to result from overproliferation of hepatocytes in liver tissue. Big contribution of the activation of the MAP kinase pathway among the HGF-signaling pathways is predicted. The results of the present examination indicate that, also in vivo, MERMUC binds to the HGF receptor causing a decrease in MAP kinase activation after HGF stimulation.

Industrial Applicability

The pharmaceutical compositions of the present invention eliminate down-regulation of HGF activity caused by the binding of c-Met and MERMUC (including multimerization of MERMUC molecules). Thus, the maximum potential biological activity of endogenous HGF or HGF (as HGF protein drug or by HGF gene therapy) exogenously supplied to the patient can be triggered.

Therefore, the use of the pharmaceutical compositions of the present invention alone or in combination with an HGF drug in disease treatment attains repair, regeneration or neogenesis of tissues and organs that is required for the prevention and treatment of various diseases. And such effect can be achieved with the required minimum dose of HGF drug (HGF protein, HGF gene, etc.). More specifically, various intractable diseases (for example, hepatic fibrosis, hepatic cirrhosis, fulminant hepatitis, viral hepatitis, acute hepatitis, chronic renal failure, renal fibrosis, pulmonary fibrosis, etc.) that are hard to treat can be treated with the present pharmaceutical compositions.

Moreover, when the pharmaceutical compositions of the present invention are applied together with an HGF drug, the dose, frequency of administration, administration period and such of the HGF drug administrated to a patient can be reduced. Accordingly, the patient's physical and mental pain can be reduced while reducing various expenses (costs borne by each of patients, medical institutes, drug makers and government agencies).

The present invention further provides methods for identifying active ingredients of the pharmaceutical compositions: (1) substances having activity to inhibit the binding between c-Met and MERMUC; (2) substances having activity to inhibit multimerization (self-association) of MERMUC molecules; or (3) substances that suppress or inhibit the expression of MERMUC. Furthermore, the present invention provides methods of testing an arbitrary substance for various activities (activity to inhibit the binding between c-Met and MERMUC, activity to inhibit multimerization of MERMUC, activity to suppress or inhibit the expression of MERMUC, and such). In addition, the present invention provides various tools (various polypeptides, fusion polypeptides, etc.) used for these methods. Using these methods and tools, the pharmaceutical compositions of the present invention can be readily discovered.

1. A pharmaceutical composition used for maintaining or enhancing cell proliferation, or formation, repair, regeneration or neogenesis of tissues, organs, blood vessels, mucosa, skeleton or nerves, which comprises a substance having activity to inhibit the binding between c-Met and MERMUC or multimerization of MERMUC, and a pharmaceutically acceptable carrier.

2. A pharmaceutical composition used for maintaining or enhancing cell proliferation, or formation, repair, regeneration or neogenesis of tissues, organs, blood vessels, mucosa, skeleton or nerves due to HGF, which comprises a substance having activity to inhibit the binding between c-Met and MERMUC or multimerization of MERMUC, and a pharmaceutically acceptable carrier.

3. The pharmaceutical composition of claim 2, wherein said HGF is an endogenous HGF of an organism.

4. The pharmaceutical composition of claim 2, wherein said HGF is an HGF drug that exogenously provides HGF to an organism.

5. The pharmaceutical composition of claim 4, wherein the HGF drug is a protein drug comprising an HGF protein.

6. The pharmaceutical composition of claim 4, wherein the HGF drug is a DNA drug comprising DNA encoding an HGF protein.

7. The pharmaceutical composition of claim 1 or 2, which is used for preventing or treating fibrosis.

8. The pharmaceutical composition of claim 7, wherein the fibrosis is hepatic, renal or pulmonary fibrosis.

9. The pharmaceutical composition of claim 1 or 2, which is used for preventing or treating hepatic cirrhosis, hepatic fibrosis or hepatitis.

10. The pharmaceutical composition of claim 9, wherein hepatitis is used for preventing or treating fulminant hepatitis, acute hepatitis or viral hepatitis.

11. The pharmaceutical composition of claim 1 or 2, which is used for preventing or treating chronic renal failure.

12. The pharmaceutical composition of claim 1 or 2, which is used for preventing or treating arteriosclerosis obliterans.

13. The pharmaceutical composition of claim 1 or 2, wherein the substance has activity to inhibit the binding between c-Met and MERMUC.

14. The pharmaceutical composition of claim 13, wherein the substance has activity to inhibit the binding between the C-terminal region of the c-Met β chain and the C-terminal region of MERMUC.

15. The pharmaceutical composition of claim 1 or 2, wherein the substance has activity to inhibit multimerization of MERMUC.

16. The pharmaceutical composition of claim 1 or 2, wherein the substance binds to MERMUC.

17. A pharmaceutical composition used for maintaining or enhancing cell proliferation, or formation, repair, regeneration or neogenesis of tissues, organs, blood vessels, mucosa, skeleton or nerves, which comprises a substance that suppresses or inhibits the expression of MERMUC and a pharmaceutically acceptable carrier.

18. The pharmaceutical composition of claim 17, wherein the substance inhibits the transcription of a gene encoding MERMUC to mRNA.

19. The pharmaceutical composition of claim 17, wherein the substance inhibits the translation of an mRNA encoding MERMUC to protein.

20. A pharmaceutical composition used for maintaining or enhancing cell proliferation, or formation, repair, regeneration or neogenesis of tissues, organs, blood vessels, mucosa, skeleton or nerves due to HGF, which comprises a substance that suppresses or inhibits the expression of MERMUC and a pharmaceutically acceptable carrier.

21. The pharmaceutical composition of claim 20, wherein the substance inhibits the transcription of a gene encoding MERMUC to mRNA.

22. The pharmaceutical composition of claim 20, wherein the substance inhibits the translation of an mRNA encoding MERMUC to protein.

23. The pharmaceutical composition of claim 20, wherein said HGF is an endogenous HGF of an organism.

24. The pharmaceutical composition of claim 20, wherein said HGF is an HGF drug that can exogenously provide HGF to an organism.

25. The pharmaceutical composition of claim 24, wherein the HGF drug is a protein drug comprising an HGF protein.

26. The pharmaceutical composition of claim 24, wherein the HGF drug is a DNA drug comprising DNA encoding an HGF protein.

27. The pharmaceutical composition of claim 17 or 20, which is used for preventing or treating fibrosis.

28. The pharmaceutical composition of claim 27, wherein the fibrosis is hepatic, renal or pulmonary fibrosis.

29. The pharmaceutical composition of claim 17 or 20, which is used for preventing or treating hepatic cirrhosis, hepatic fibrosis or hepatitis.

30. The pharmaceutical composition of claim 29, wherein hepatitis is used for preventing or treating fulminant hepatitis, acute hepatitis or viral hepatitis.

31. The pharmaceutical composition of claim 17 or 20, which is used for preventing or treating chronic renal failure.

32. The pharmaceutical composition of claim 17 or 20, which is used for preventing or treating arteriosclerosis obliterans.

33. A polypeptide comprising a partial amino acid sequence of the full-length amino acid sequence of MERMUC, and having activity to bind c-Met.

34. The polypeptide of claim 33, wherein said partial amino acid sequence corresponds to whole or a portion of a cytoplasmic region of MERMUC.

35. The polypeptide of claim 34, wherein said whole or a portion of a cytoplasmic region comprises whole or a portion of a leucine repeat region in the C-terminal region of MERMUC.

36. The polypeptide of claim 33, wherein said MERMUC is human MERMUC.

37. The polypeptide of claim 33, which comprises the amino acid sequence of SEQ ID NO: 15.

38. A polypeptide comprising a partial amino acid sequence of the full-length amino acid sequence of c-Met, and having activity to bind MERMUC.

39. The polypeptide of claim 38, wherein said partial amino acid sequence corresponds to whole or a portion of a cytoplasmic region of the c-Met β chain.

40. The polypeptide of claim 39, wherein said whole or a portion of a cytoplasmic region comprises whole or a portion of a C-terminal region of c-Met comprising one or more tyrosine residues that undergo self-phosphorylation.

41. The polypeptide of claim 38, wherein said c-Met is human c-Met.

42. The polypeptide of claim 38, which comprises the amino acid sequence of SEQ ID NO: 5.

43. A polypeptide comprising a partial amino acid sequence of the full-length amino acid sequence of MERMUC, and comprising the activity to bind MERMUC.

44. The polypeptide of claim 43, wherein said partial amino acid sequence corresponds to whole or a portion of a cytoplasmic region of MERMUC.

45. The polypeptide of claim 43, wherein said MERMUC is human MERMUC.

46. A fusion polypeptide represented by the general formula W—X—Y (wherein, W is absent, or the full-length amino acid sequence or a portion of a protein other than MERMUC; X is the full-length amino acid sequence of MERMUC or a portion thereof, and Y is the full-length amino acid sequence or a portion of a protein other than MERMUC).

47. The fusion polypeptide of claim 46, wherein the protein Y is β-galΔα or β-galΔω.

48. The fusion polypeptide of claim 47, wherein said β-galΔα comprises the amino acid sequence of SEQ ID NO: 16.

49. The fusion polypeptide of claim 47, wherein said β-galΔω comprises the amino acid sequence of SEQ ID NO: 17.

50. The fusion polypeptide of claim 46, wherein said portion of the full-length amino acid sequence of MERMUC has activity to bind c-Met.

51. The fusion polypeptide of claim 46, wherein said portion of the full-length amino acid sequence of MERMUC comprises whole or a portion of a cytoplasmic region of MERMUC.

52. The fusion polypeptide of claim 51, wherein said portion of the full-length amino acid sequence of MERMUC comprises whole or a portion of a leucine repeat region of the C-terminal region of MERMUC.

53. The fusion polypeptide of claim 46, wherein said MERMUC is human MERMUC.

54. The polypeptide of claim 46, wherein said X comprises the amino acid sequence of SEQ ID NO: 15.

55. The polypeptide of claim 46, wherein the protein W comprises the extracellular region of Fas.

56. A fusion polypeptide represented by the general formula Z-Y (wherein Z is the full-length amino acid sequence of c-Met or a portion thereof; and Y is the full-length amino acid sequence or a portion of a protein other than c-Met).

57. The fusion polypeptide of claim 52, wherein said protein is β-galΔω or β-galΔω.

58. The fusion polypeptide of claim 57, wherein said β-galΔω comprises the amino acid sequence of SEQ ID NO: 16.

59. The fusion polypeptide of claim 57, wherein said β-galΔω comprises the amino acid sequence of SEQ ID NO: 17.

60. The fusion polypeptide of claim 56, wherein said portion of the full-length amino acid sequence of c-Met has activity to bind MERMUC.

61. The fusion polypeptide of claim 56, wherein said portion of the full-length amino acid sequence of c-Met comprises whole or a portion of a cytoplasmic region of the c-Met β chain.

62. The fusion polypeptide of claim 56, wherein said portion of the full-length amino acid sequence of c-Met comprises whole or a portion of a C-terminal region of c-Met comprising one or more tyrosine residues that undergo self-phosphorylation.

63. The fusion polypeptide of claim 56, wherein said c-Met is human c-Met.

64. The fusion polypeptide of claim 56, wherein said Z comprises the amino acid sequence of SEQ ID NO: 5.

65. A method of testing a substance for the presence or extent of activity to inhibit the binding between c-Met and MERMUC, which comprises the step of comparing the presence or extent of the binding between c-Met and MERMUC in the presence of a test substance to the extent of the binding between c-Met and MERMUC in the absence of the test substance.

66. A method of identifying a substance that has activity to inhibit the binding between c-Met and MERMUC, which comprises the step of comparing the presence or extent of the binding between c-Met and MERMUC in the presence of a test substance to the extent of the binding between c-Met and MERMUC in the absence of the test substance.

67. A method of testing for the presence or extent of activity of a substance to inhibit the binding between c-Met and MERMUC, which comprises the steps of:
(a) preparing a cell that is designed to coexpress c-Met and MERMUC, and allow detection of the binding between said c-Met and MERMUC;
(b) culturing the cell obtained in step (a) in the presence of HGF while in the absence of test substance, and then measuring the extent of the binding between c-Met and MERMUC in the cell;
(c) culturing the cell obtained in step (a) in the presence of HGF and a test substance, and then measuring the extent of the binding between c-Met and MERMUC in the cell; and
(d) comparing the measurement result of step (b) with that of step (c).

68. The method of claim 67, wherein said MERMUC is the fusion polypeptide of any one of claims 46 to 54.

69. The method of claim 67, wherein said MERMUC is the fusion polypeptide of any one of claims 47 to 49.

70. The method of claim 67, wherein said c-Met is the fusion polypeptide of any one of claims 56 to 64.

71. The method of claim 67, wherein said c-Met is the fusion polypeptide of any one of claims 57 to 59.

72. A method of identifying a substance that has activity to inhibit the binding between c-Met and MERMUC, which comprises the steps of:
(a) preparing a cell that is designed to coexpress c-Met and MERMUC, and allow detection of the binding between said c-Met and MERMUC bind;
(b) culturing the cell obtained in step (a) in the presence of HGF while in the absence of test substance, and then measuring the extent of the binding between c-Met and MERMUC in the cell;
(c) culturing the cell obtained in step (a) in the presence of HGF and a test substance, and then measuring the extent of the binding between c-Met and MERMUC in the cell; and
(d) comparing the measurement result of step (b) with that of step (c).

73. The method of claim 72, wherein said MERMUC is the fusion polypeptide of any one of claims 46 to 54.

74. The method of claim 72, wherein said MERMUC is the fusion polypeptide of any one of claims 47 to 49.

75. The method of claim 72, wherein said c-Met is the fusion polypeptide of any one of claims 56 to 64.

76. The method of claim 72, wherein said c-Met is the fusion polypeptide of any one of claims 57 to 59.

77. A method of testing a substance for the presence or extent of activity to inhibit multimerization of MERMUC, which comprises the step of comparing the presence or extent of multimerization of MERMUC in the presence of a test substance to the extent of multimerization of MERMUC in the absence of the test substance.

78. A method for identifying a substance having activity to inhibit multimerization of MERMUC, which comprises the step of comparing the presence or extent of multimerization of MERMUC in the presence of a test compound to the extent of multimerization of MERMUC in the absence of the test substance.
